SEQUENCE LISTING (13 sequences)

SEQ ID NO: 1 - DNA, 81 bp, Mycobacterium tuberculosis
  source 1..81: mol_type = other DNA
ggcaccagccagctgagccaattcatggaccagaacaacccgctgtcggggttgacccacaagcgccgactgtcggcgctg

SEQ ID NO: 2 - DNA, 77 bp, Escherichia coli
  source 1..77: mol_type = other DNA
acaagaaagggatcttcactcgcgaccgcaaaccgaagtcggcggcttttctgctgcaaaaacgctggactggcatg

SEQ ID NO: 3 - DNA, 200 bp, synthetic construct
  source 1..200: mol_type = other DNA
atctggtcggccccgaacccgaggctgctccgctggagcagatgggcttgggctggaagagctcgtatggcaccggaaccggtaaggacgcgatcaccagcggcatcgaggtcgtatggacgaacaccccgacgaaatgggacaacagtttcctcgagatcctgtacggctacgagtgggagctgacgaagagccctgct

SEQ ID NO: 4 - DNA, 15 bp, unidentified
  source 1..15: mol_type = other DNA
gcgatcaccagcggc

SEQ ID NO: 5 - DNA, 15 bp, synthetic construct
  source 1..15: mol_type = other DNA
gcgatcaccaccggc

SEQ ID NO: 6 - DNA, 15 bp, synthetic construct
  source 1..15: mol_type = other DNA
gcgatcaccaacggc

SEQ ID NO: 7 - DNA, 15 bp, synthetic construct
  source 1..15: mol_type = other DNA
gcgatcaccatcggc

SEQ ID NO: 8 - DNA, 15 bp, synthetic construct
  source 1..15: mol_type = other DNA
gcgatcaccagaggc

SEQ ID NO: 9 - DNA, 15 bp, synthetic construct
  source 1..15: mol_type = other DNA
gcgatcaccggcggc

SEQ ID NO: 10 - DNA, 15 bp, synthetic construct
  source 1..15: mol_type = other DNA
gcgatcaccctcggc

SEQ ID NO: 11 - DNA, 15 bp, synthetic construct
  source 1..15: mol_type = other DNA
gtgatcaccaccggc

SEQ ID NO: 12 - DNA, 15 bp, synthetic construct
  source 1..15: mol_type = other DNA
gcgatcaccaccgac

SEQ ID NO: 13 - DNA, 15 bp, synthetic construct
  source 1..15: mol_type = other DNA
gtgatcaccaccgac

(19) United States
(12) Patent Application Publication
(10) Pub. No.: US 2024/0318229 A1
(43) Pub. Date: Sep. 26, 2024

(54) COMPARATIVE TEMPLATING FOR DIRECT SEQUENCE VARIATION DETECTION

(71) Applicant: Vanderbilt University, Nashville, TX (US)

(72) Inventor: Frederick R. HASELTON, Nashville, TN (US)

(73) Assignee: Vanderbilt University, Nashville, TX (US)

(21) Appl. No.: 18/586,089

(22) Filed: Feb. 23, 2024

Related U.S. Application Data
(60) Provisional application No. 63/486,769, filed on Feb. 24, 2023.

(51) Int. Cl. C12Q 1/6809; C12Q 1/6844; C12Q 1/689

(52) U.S. Cl. CPC C12Q 1/6809; C12Q 1/6844; C12Q 1/689

(57) ABSTRACT

Variation in a single nucleotide of a target or template nucleic acid may be significant in many ways. For infectious disease, many drug resistance mutations are known and a simple means to confirm their absence would more quickly match a patient with an infection with the most effective treatment. This disclosure provides for a simple, low resource strategy allowing the detection of single nucleotide variation using only low-cost diagnostic methods already available in many locations. A key to this approach is the use of left-handed DNA (L-DNA) as a comparator molecule for D-DNA targets. L-DNA provides numerous additional advantages such as low cost and stability.

### PRIORITY CLAIM

This application claims benefit of priority to U.S. Provisional Application Ser. No. 63/486,769, filed Feb. 24, 2023, the entire contents of which are hereby incorporated by reference.

### STATEMENT REGARDING FEDERALLY FUNDED RESEARCH

This invention was made with government support under grant no. R21AI152497 and R01AI135937, awarded by the National Institutes of Health. The government has certain rights in the invention.

### REFERENCE TO A SEQUENCE LISTING

This application contains a Sequence Listing XML, which has been submitted electronically and is hereby incorporated by reference in its entirety. Said XML Sequence Listing, created on Feb. 23, 2024, is named VBLTP0334US.xml and is 12,520 bytes in size.

### BACKGROUND

### 1. Field of the Disclosure

The present disclosure relates generally to fields of molecular biology, nucleic acid chemistry, manufacturing and diagnostics. More particularly, the disclosure relates to the use of L-DNA molecules to identify sequence differences in nucleic acid amplification products.

### 2. Background

In regions with large populations of people living with infectious disease, like South Africa, the lack of simple and cost-effective methods to detect and characterize such infections continues to impede treatment management and the spread of infection. For example, sequencing of resistant TB strains has shown that resistance to the most widely used oral treatment, rifampicin, can be conferred by any one of many single nucleotide changes in an 81 base pair region of the TB genome. Culture methods that take weeks to months to perform and also delay the start of the most effective treatment remain the standard in most resource constrained settings.

Melt analysis is another method used to detect differences between a rifampicin susceptible wild-type sequence and a drug resistant sequence that contains only a single base difference. Unfortunately, only high resource settings can afford the calibrated instrumentation and staff technical expertise to implement the complex reagent designs now required to make this measurement. A less complex, but robust and sensitive reagent design, would make melt analysis more available by enabling implementation with simpler PCR instrumentation. As such, there remains a significant unmet need in this area.

### SUMMARY

Thus, in accordance with the present disclosure, there is provided a method of detecting sequence variation in a test D-DNA nucleic acid as compared to reference D-DNA sequence comprising (a) providing an L-DNA sequence that is identical to the reference D-DNA sequence except for its stereochemistry; (b) obtaining a melt curve for said L-DNA sequence; (c) obtaining a melt curve for said test D-DNA sequence under conditions identical to step (b); and (d) comparing the melt curves of step (b) and step (c), wherein a difference in melt curves indicates that said test D-DNA does not have the same sequence as said reference D-DNA sequence. The L-DNA sequence and the test D-DNA sequence may be detected using an intercalating dye and/or a dye/quencher pair or by hyperchromicity. The intercalating dye may be selected from the group consisting of SYBR® Gold, SYBR® Green, EvaGreen, SYTO 82, SYTO 64, SYTO 9, and LCGreen dyes. Obtaining a melt curve may comprise heat separation of said L-DNA and/or said test D-DNA sequence. Steps (b) and (c) may be performed simultaneously in the same reaction mixture.

The test D-DNA may be a template dependent amplification product. Multiple distinct L-DNA sequences with multiple melt properties may be used together. The L-DNA may be end-labeled one strand with a dye and end-labeled with quencher on the other strand, or the L-DNA and D-DNA are labeled with an intercalating dye that is not impacted by the quencher. The D-DNA may be a template-dependent amplification product. The L-DNA may be present in template-dependent reaction mixture. A single intercalating dye may be used for both L-DNA and D-DNA

The melt properties for the L-DNA may be assessed prior to mixture with D-DNA, and the D-DNA may be added to the L-DNA mixture and a second melt property assessment is performed. Obtaining a melt curve for the L-DNA and/or the D-DNA may comprise template dependent amplification. The method may further comprise quantitating D-DNA products. The L-DNA may be one that has the same sequence as a natural D-DNA that is unique to a drug susceptible or resistant pathogen, such as a drug resistant bacterium or virus. The melt temperature for L-DNA may be adjusted to approximate the melt temperature of the D-DNA using the ratio of the forward:reverse strands of L-DNA. The pathogen may be Mycobacterium tuberculosis, and the sequence may be ggcaccagccagctgagccaattcatggaccagaacAACCC GCTgtcggggttgacccacaagcgccgactgtcgg cgctg (SEQ ID NO: 1).

Also provides is a kit comprising an L-DNA and at least one detectable moiety. The L-DNA may be a single-stranded sequence or a double-stranded sequence. The L-DNA may have the same sequence as a naturally occurring D-DNA sequence, or the L-DNA may have less than 10% base differences as compared to the naturally occurring D-DNA sequence. The kit may further comprise standard double-stranded sequences L-DNA and/or D-DNA sequence that melt at known relative temperatures. The L-DNA may comprise partial sequence pairs for molecular beacon detection. The kit may further comprise a melt detection reagent, such as an intercalating dye that intercalate within both L-DNA and D-DNA, an intercalating dye that preferentially intercalates within L-DNA or D-DNA, a fluorescence-quencher pair, or an end-labelling reagent, such as wherein the end-labelling reagent is disposed on the 3′ and 5′ ends of two L-DNA complementary strands whose interaction is detectable. The melt detection agent may be one or more modified L-DNA bases incorporated into said L-DNA that exhibit electrical sensing changes during melting. The kit may further comprise one or more additives that modify melting differences, such as salts or betaine. The kit may further comprise (a) a device to perform heating of mixture over a range of room temperature to 99° C.; (b) a device to measure fluorescence; (c) a device to measure absorbance of light (e.g., a UV light emitting diode); (d) a device to measure electrical changes of solution; (e) a microprocessor (e.g., raspberry PI); and/or (f) a “smart” device with sensing capabilities. The kit may further comprise instructions for using the kit and its components, such as in a method according to the present disclosure.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

In another embodiment, there is provided a kit comprising an L-DNA and at least one detectable moiety. The L-DNA may be a single-stranded sequence or a double-stranded sequence. The L-DNA may have the same sequence as a naturally occurring D-DNA sequence, or wherein the L-DNA may have less than 10% base differences as compared to the naturally occurring D-DNA sequence. The kit may further comprise standard double-stranded sequences L-DNA and/or D-DNA sequence that melt at known relative temperatures. The L-DNA may comprise partial sequence pairs for molecular beacon detection.

The kit may further comprise a melt detection reagent, such as an intercalating dye that intercalate within both L-DNA and D-DNA, an intercalating dye that preferentially intercalates within L-DNA or D-DNA, a fluorescence-quencher pair, or an end-labelling reagent, such as wherein the end-labelling reagent is disposed on the 3′ and 5′ ends of two L-DNA complementary strands whose interaction is detectable. The melt detection agent may be one or more modified L-DNA bases incorporated into said L-DNA that exhibit electrical sensing changes during melting.

The kit may further comprise one or more additives that modify melting differences, such as salts or betaine. The kit may further comprise:
(a) a device to perform heating of mixture over a range of room temperature to 99° C.;
(b) a device to measure fluorescence;
(c) a device to measure absorbance of light (e.g., a UV light emitting diode);
(d) a device to measure electrical changes of solution;
(e) a microprocessor (e.g., raspberry PI); and/or
(f) a “smart” device with sensing capabilities.
   The kit may also further comprise instructions for using the kit and its components, such as in a method according to the present disclosure.

It is contemplated that any method or composition described herein can be implemented with respect to any other method or composition described herein. Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein. The patent or application file contains at least one drawing executed in color. Copies of this patent or patent application publication with color drawing(s) will be provided by the Office upon request and payment of the necessary fee.

FIG. 1. High resolution melt analysis using an internal L-DNA comparator. The upper panel shows the fluorescence as a function of temperature for the pre-PCR melt (red, lower line) and the post-PCR (blue, upper line). The lower panel compares the derivatives of these curves. For this 5-base mutation, there is an easily detectable shift in the derivatives, which, based on the instrument calibration is about 3° C.

FIG. 2. Single-tube melt comparison of an L-DNA comparator (green) with a wild-type (blue) for wild-type sample (top) or the C→T mutation (bottom).

FIG. 3. L-DNA alternate melt detection method. Traditional intercalating dyes (upper panel) show an increase in fluorescence when “intercalating” within double-stranded DNA and show a decrease fluorescence as temperature increases. Alternatively, End-labelled L-DNA (lower panel) decrease fluorescence when strands are hybridized and show an increase fluorescence when temperature increases.

FIG. 4. Comparison of melt curves between 8° and 90° C. for SYBR® green intercalating dye (top, blue) and end-labelled L-DNA sequence (bottom, orange). Note that though the derivatives are inverted, reflecting the change in fluorescence seen with the two methods, their respective peaks are both calculated as 85.5° C. by traditional melt analysis.

FIG. 5. Molecular beacon design for detecting melt characteristics of D-DNA components (left) with L-DNA (right) in the same reaction. Molecular beacons fluoresce when bound to the PCR amplicon structures. Two fluorescent labels and quenchers are used to provide differentiation of the D-DNA structures on the left with the comparator L-DNA structures shown on the right.

FIG. 6. L-DNA (right) is a left-helical enantiomer of D-DNA (left).

FIG. 7. Wild-type drug-susceptible katG sequence compared to nine variants with INH-resistance-related mutations. (SEQ ID NOS: 4-13)

FIG. 8. Representative PCR product derivative melt plots of standard HRM. Each curve is labeled with the known synthetic sample classification, including wild-type katG, clincially-relevant katG variants, and no template control (NTC). The nine selected variants had a Tm spread of 2.43° C., offering easy and challenging classification cases against wild-type. The inventors selected nine out of ten possible variants in the primer-bounded 56-base amplicon, among 250+ INH-resistance-related mutations in the katG gene overall. Dashed lines indicate PCR product Tm.

FIGS. 9A-B. Melting temperatures of (FIG. 9A) wild-type drug-susceptible katG and (FIG. 9B) S315T samples and their associated thermal profiles in the top left and top right quadrants, respectively, of a 96-well plate.

FIG. 10. Representative PCR product (red) and internal comparator L-DNA (green) derivative melt plots of L-DNA-based HRM within single samples. Each curve is labeled with the known synthetic sample classification, including wild-type katG, clincially-relevant katG variants, and no template control (NTC). The nine selected variants had a Tm spread of 88.06 sec, offering easy and challenging classification cases against wild-type. Nine out of ten possible variants were selected in the primer-bounded 56-base amplicon, among 250+ INH-resistance-related mutations in the katG gene overall. Dashed lines indicate DNA PCR product tm (red) and L-DNA tm (green).

FIG. 11. The difference in melt times between L-DNA double strand and PCR products for wild-type samples and 9 synthetic variants. These differences were used to correctly classify samples into drug susceptible and not susceptible groups.

FIGS. 12A-B. (FIG. 12A) Representative melt curve derivatives for L-DNA-based HRM reactions with double-stranded L-DNA at 1:1, 1:2, and 1:3 ratio of forward to reverse L-DNA strands with 1×1011 forward strand copies and 1×1011, 2×1011, and 3×1011 reverse strand copies per reaction, respectively. (FIG. 12B) There is a positive linear relationship between number of L-DNA reverse strand copies and L-DNA tm measured via fluorophore-quencher signal. This relationship hypothesized that an L-DNA forward to reverse strand ratio of 1:2.79 (1×1011 forward strand copies and 2.79×1011 reverse strand copies per reaction) would match drug-susceptible L-DNA tm to that of drug-susceptible DNA PCR product average tm of 695 sec.

FIG. 13. Melt curve derivatives for L-DNA-based HRM reactions with 1:1 double-stranded L-DNA at 4×1011, 2×1011, 1×1011 copies per strand (forward and reverse) per reaction. As total L-DNA concentration decreased, both intercalator signal (lower line) and fluorophore-quencher signal (upper line) decreased in magnitude. End-labeled strands retained tm identification even when strand concentration decreased.

### DESCRIPTION OF ILLUSTRATIVE EMBODIMENTS

As discussed above, low resource but highly sensitive assays to detect subtle changes, including single nucleotide substitutions and deletions, are greatly in need. The inventor here proposes a (potentially) single-tube comparator method based on the inclusion of an internal synthetic comparator with melt characteristics identical to the wild-type PCR product to simplify reagent design and melt analysis measurements. Preliminary data suggest that melt analysis based on this approach has the single-base sensitivity required for the detection of rifampicin susceptibility. In a particular application, the sequence sensitivity of this approach for single nucleotide polymorphisms can be applied to the TB rifampicin resistance-determining region. The reagent design, based on L-DNA, makes melt analysis approach possible in resource-constrained settings through its less expensive reagent design that simplifies PCR testing. Because the method is sequence-based, it is anticipated that it can be easily modified for use in other diagnostic scenarios where knowledge of small changes in a known drug-susceptible sequence impacts the treatment management decision.

L-DNA has a number of features that make it ideal as a comparator for D-DNA sequences. First, L-DNA is biologically inert and resistant to degradation. There are no natural nucleases that target and degrade L-DNA (Urata et al., 1991). Second, L-DNA is easily and inexpensively manufactured in large quantities. L-DNAs are synthesized using the same phosphoramidite chemistry that is used to synthesize DNA oligonucleotides. Third, and perhaps most significantly, L-DNA has by design all of the physical chemical properties of its D-DNA counterpart but does not interact with the D-DNA present in the same solution, including the base melt temperature characteristics. These properties enable the use of L-DNA templates as additives to a PCR reaction mix to serve as a perfect wild-type comparator during melt analysis.

Collectively, the data shown in this disclosure support the hypothesis that adding L-DNA improves standard HRM. In the context of drug susceptibility screening for the TB drug INH it improved the classification of known synthetic variants compared to standard HRM. L-DNA-based HRM designs can be applied to other clinical applications may include screening for SNP-induced diseases, disease variants, or drug-resistance by adapting the key design features identified in this study for other screening applications. These key requirements include (1) L-DNA is designed as an exact sequence match to the PCR amplicon of interest (e.g., consensus wild-type or alternatively a mutated sequence of interest), (2) the L-DNA is made as long as possible within synthesis limitations (acknowledging that reagent costs are increased and SNP-induced tm changes are reduced with longer sequences), and (3) L-DNA is end-labeled to achieve differential detection between L-DNA and D-DNA and avoid intercalation crosstalk. By including a constant melt comparator in every sample, L-DNA-based HRM improves the classification of PCR melt products by quantifying within sample differences between the constant L-DNA comparator and an unknown PCR product.

These and other aspects of the disclosure are described in detail below.

### I. L-DNA

L-DNA is the enantiomer of the natural D-DNA. L-Deoxyribose, the sugar backbone of L-DNA, is mirror of natural D-deoxyribose and has not been found in nature despite the fact that other L-sugars (e.g., L-arabinose, L-lyxose, L-galactose, L-sorbose, and L-xylulose) do exist in nature. L-DNA can hybridize with complementary L-DNA sequences via classical Watson-Crick base-pairing, forming an L-DNA duplex much like a D-DNA duplex, except that the L-DNA duplex is a left-handed B-helix, a mirror structure of the right-handed B-helix by D-DNA. Importantly, however, L-DNA does not hybridize with the complementary sequence on D-DNA backbones or D-RNA at physiological temperatures.

Given that L-DNA is the enantiomer of D-DNA, the chemical reactivity of L-DNA is identical to D-DNA if the reactant is achiral. The biological reactivity of L-DNA with the chiral proteins and enzymes, however, is completely distinguishable from that of D-DNA. For example, L-DNA is not susceptible to natural DNA-modifying enzymes (e.g., ligases, polymerases and nucleases that can easily modify or degrade natural D-DNA). Taking advantage of these properties, L-DNA has been used to build intracellular nano-sensors as well as aptamers exhibit an enhanced serum stability. L-DNA has also been employed to construct a self-assembled DNA tetrahedral nanostructure showing high cellular uptake. L-DNA has also been adapted to PCR (see Adams et al., 2016) and used as a biostable DNAzyme with achiral metal ions (see Cui et al., 2016). Various aspects and uses of L-DNA is reviewed in Young et al., 2019. It also has been shown to interact with intercalating dyes just the same as D-DNA.

### II. L-DNA SYNTHESIS

The first chemical synthesis of L-DNA oligonucleotides was reported by Anderson et al., (1984) who prepared L-dU 18-mer by using the triester approach (Reese, 1978; Narang et al., 1980). A more recent publication describes synthesis using either α-L pr β-L nucleotide unnits and covalently linked to an acridine derivative (Asseline et al., 1991).

### III. MELT CURVES

### A. Melt Curve Analysis

Melting curve analysis is an assessment of the dissociation characteristics of double-stranded DNA during heating. As the temperature is raised, the double strand begins to dissociate leading to a rise in the absorbance of light at 260 nm and hyperchromicity. The temperature at which 50% of DNA is denatured is known as the melting temperature. Measurement of melting temperature can help characterize a PCR product by just studying the melting temperature. This is because every ds DNA sequence has a specific melting temperature.

The information gathered can be used to infer the presence and identity of single-nucleotide polymorphisms (SNP). This is because G-C base pairing have 3 hydrogen bonds between them while A-T base pairs have only 2. DNA with a higher G-C content, whether because of its source (G-C contents: E. coli 0.50, M. luteus 0.72, poly d(AT) 0.00) or, as previously mentioned, because of SNPs, will have a higher melting temperature than DNA with a higher A-T content. Base interactions with neighboring bases also affect the melt temperature.

The information also gives vital clues to a molecule's mode of interaction with DNA. Molecules such as intercalators slot in between base pairs and interact through pi stacking. This has a stabilizing effect on DNA's structure which, along with “nearest neighbors” effects, can lead to a rise in its melting temperature. Likewise, increasing salt concentrations helps diffuse negative repulsions between the phosphates in the DNA's backbone. This also leads to an increase in the DNA's melting temperature. Conversely, pH can have a negative effect on DNA's stability which may lead to a lowering of its melting temperature (Botezatu et al., 2011).

The energy required to break the base-base hydrogen bonding between two strands of DNA is dependent on their length, GC content and their complementarity and “nearest neighbors.” By heating a reaction-mixture that contains double-stranded DNA sequences and measuring dissociation against temperature, these attributes can be inferred. Originally, strand dissociation was observed using UV absorbance measurements, but techniques based on fluorescence measurements are now the most common approach.

The temperature-dependent dissociation between two DNA-strands can be measured using a DNA-intercalating fluorophore such as SYBR green, EvaGreen or fluorophore-labelled DNA probes. In the case of SYBR green (which fluoresces 1000-fold more intensely while intercalated in the minor groove of two strands of DNA), the dissociation of the DNA during heating is measurable by the large reduction in fluorescence that results. Alternatively, juxtapositioned probes (one featuring a fluorophore and the other, a suitable quencher; often referred to as “molecular beacons”) can be used to determine the complementarity of the probe to the target sequence when the strands are separated.

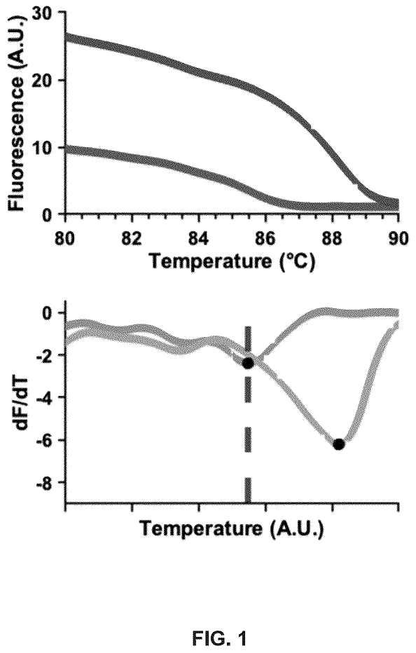

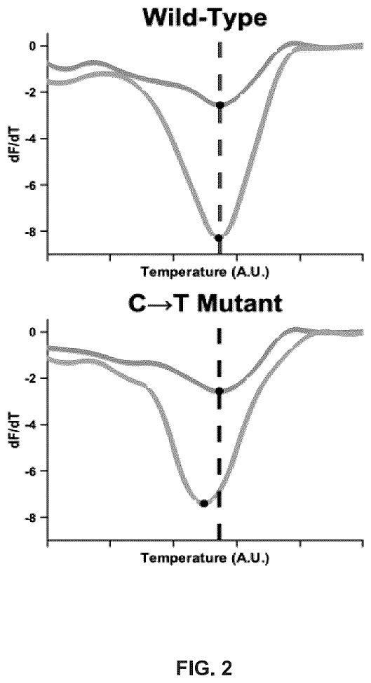

The graph of the negative first derivative of the melting-curve may make it easier to pin-point the temperature of dissociation (defined as 50% dissociation), by virtue of the peaks thus formed. See FIG. 1.

SYBR Green enabled product differentiation in the LightCycler in 1997. Hybridization probes (or FRET probes) were also demonstrated to provide very specific melting curves from the single-stranded (ss) probe-to-amplicon hybrid. Idaho Technology and Roche have done much to popularize this use on the LightCycler instrument.

Since the late 1990s product analysis via SYBR Green, other double-strand specific dyes, or probe-based melting curve analysis has become nearly ubiquitous. The probe-based technique is sensitive enough to detect single-nucleotide polymorphisms (SNP) and can distinguish between homozygous wildtype, heterozygous and homozygous mutant alleles by virtue of the dissociation patterns produced. Without probes, amplicon melting (melting and analysis of the entire PCR product) was not generally successful at finding single base variants through melting profiles. With higher resolution instruments and advanced dyes, amplicon melting analysis of one-base variants is now possible with several commercially available instruments. For example: Applied Biosystems 7500 Fast System and the 7900HT Fast Real-Time PCR System, Idaho Technology's LightScanner (the first plate-based high resolution melting device), Qiagen's Rotor-Gene instruments, and Roche's LightCycler 480 instruments. Many research and clinical examples exist in the literature that show the use of melting curve analysis to obviate or complement sequencing efforts, and thus reduce costs.

While most real-time PCR machines have the option of melting curve generation and visualization, the level of analysis and software support varies. In general, they do not have high resolution capabilities. High Resolution Melt (known as either Hi-Res Melting, or HRM) is the advancement of this general technology and has begun to offer higher sensitivity for SNP detection within an entire dye-intercalating amplicon. It is less expensive and simpler in design to develop probeless melting curve systems. However, for genotyping applications, where large volumes of samples must be processed, the cost of development may be less important than the total throughput and ease of interpretation, thus favoring sequence probe-based genotyping methods.

### B. Melt Property Detection Formats

It is necessary for the purpose of the methods described herein to have sufficient amounts of both the L-DNA comparator and the D-DNA unknown. The user can control L-DNA by simply adding as much as needed, the D-DNA unknown cannot be as easily manipulated. One way to ensure sufficiently high copy number to enable detection during heating that suddenly separates the double strands at a particular temperature is by PCR, which can make numerous copies of the unknown. However, only the primer sites flanking the region of interest are guaranteed to be of a known sequence. In contrast the sequence between the primers may vary as it is copied from the template target sequence. The melt approaches applied here detect whether that unknown internal sequence between the primers is an exact match for the expected sequence.

In order to perform a comparison between an unknown target (e.g., a PCR amplicon) and the L-DNA comparator, a way to independently detect the melt properties of each is required. One way employs traditional melt analysis performed using intercalating dyes that fluoresce much more brightly when bound or intercalated between two strands of hybridized DNA. Based on current studies, all known intercalating dyes intercalate into D-DNA and L-DNA equally. For example, SYBR® green intercalates and can be used to detect the separation of two complementary strands as a function of temperature for D-DNA, but also for L-DNA as well. In this method, the template L-DNA is added to a PCR reaction in low concentration along with a dye (e.g., SYBR® green) and an initial melt analysis curve is obtained. Importantly, before the PCR reaction has been started, the reaction does not contain any detectable double-stranded amplicon product. Thus, this initial melt analysis only reflects the characteristics of the L-DNA wild-type template. After the PCR reaction has been completed, the PCR reaction would typically contain more than 1012 copies of amplicon in D-DNA form. A second melt curve is then performed and compared to the initial melt curve obtained with the low concentration of the L-DNA wild-type template.

A second method compares the intercalating dye to a fluorescent probe on one strand of the L-DNA duplex and a quencher on the complementary strand to do a comparison simultaneously in two PCR fluorescence channels. In this approach, the separation of the labelled strands shows a decrease in the fluorescence signals associated with the enantiomeric structures by comparing the melt properties obtained with the intercalating dye to that obtained with the L-DNA end labeling. For example, the L-DNA wild-type template is labelled with Texas Red dye on one strand and a quencher on the opposite strand; then SYBR® green is added as the intercalating dye. This allows comparison between the Texas Red channel for L-DNA alone and the SYBR® green channel for all double-stranded structures. By selecting and controlling the concentration of the two dyes, the L-DNA end-label signal can be used to determine the melt characteristics of each structure.

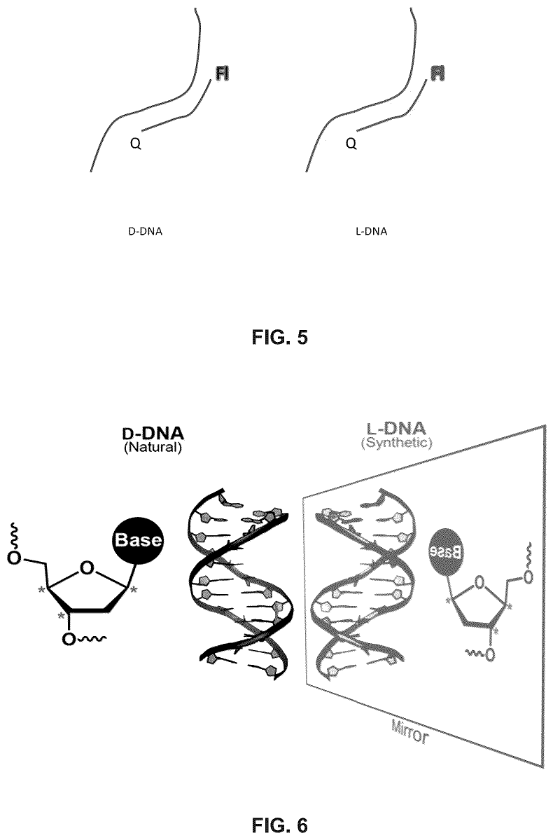

A third method is based on asymmetric PCR amplification. In this method the primer ratio is adjusted so that when one primer is exhausted the remaining primer produces an excess of one of the PCR amplicon strands. Changes in the sequence of this excess strand is determined by comparing the melt of a targeted D-DNA molecular beacon (added to the reaction) to this excess D-DNA strand in comparison to L-DNA reagents added as equivalent molecular beacon and an analog of the excess D-DNA strand. This is pictured in FIG. 5.

A fourth approach, using some of the elements of the first two methods, is to simply include several L-DNA “standard” structures with known melt characteristics that span the unknown nucleic acid sample melt characteristics. In this design, the melt characteristics of the unknown nucleic acid are determined in reference to L-DNA standards. This could be implemented by spiking the L-DNA standards into the reaction before the PCR reaction. Since these structures are made from L-DNA, they do not interact with or interfere with the enzymes or other biological structures present in the PCR reaction. In this design, the L-DNA structures produce known characteristic melt peaks obtained by existing methods that determine the derivative by fluorescence versus temperature. This scale is then overlaid with the melt peak of the unknown nucleic acid to determine the melt characteristics of the unknown. This is completely analogous to how “standards” are included within gel electrophoresis of DNA structures. In gels, the standards with known DNA characteristics (usually length) produce a pattern in a separate lane of the gel and this pattern of knowns is compared with the unknown nucleic acid.

### IV. OTHER TECHNIQUES AND REAGENTS

### A. Template Dependent Nucleic Acid Amplification

A number of template dependent processes are available to amplify the sequences present in a given template sample. One of the best-known amplification methods is the polymerase chain reaction (referred to as PCR™) which is described in detail in U.S. Pat. Nos. 4,683,195, 4,683,202 and 4,800,159, and in Innis et al., 1990, each of which is incorporated herein by reference in its entirety.

Briefly, in PCR™, two primer sequences are prepared that are complementary to regions on opposite complementary strands of the sequence of interest. An excess of deoxynucleoside triphosphates is added to a reaction mixture along with a DNA polymerase, e.g., Taq polymerase. If the sequence of interest is present in a sample, the primers will bind to the sequence of interest and the polymerase will cause the primers to be extended along the sequence of interest by adding on nucleotides. By raising and lowering the temperature of the reaction mixture, the extended primers will dissociate from the sequence of interest to form reaction products, excess primers will bind to the sequence of interest and to the reaction products and the process is repeated.

A reverse transcriptase PCR™ amplification procedure may be performed in order to quantify the amount of mRNA amplified. Methods of reverse transcribing RNA into cDNA are well known and described in Sambrook et al. (1989). Alternative methods for reverse transcription utilize thermostable, RNA-dependent DNA polymerases. These methods are described in WO 90/07641 filed Dec. 21, 1990. Polymerase chain reaction methodologies are well known in the art.

Another method for amplification is the ligase chain reaction (“LCR”), disclosed in EPO No. 320 308, incorporated herein by reference in its entirety. In LCR, two complementary probe pairs are prepared, and in the presence of the target sequence, each pair will bind to opposite complementary strands of the target such that they abut. In the presence of a ligase, the two probe pairs will link to form a single unit. By temperature cycling, as in PCR™, bound ligated units dissociate from the target and then serve as “target sequences” for ligation of excess probe pairs. U.S. Pat. No. 4,883,750 describes a method similar to LCR for binding probe pairs to a target sequence.

Qbeta Replicase, described in PCT Application No. PCT/US87/00880, may also be used as still another amplification method in the present disclosure. In this method, a replicative sequence of RNA that has a region complementary to that of a target is added to a sample in the presence of an RNA polymerase. The polymerase will copy the replicative sequence that can then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present disclosure (Walker et al., 1992).

Strand Displacement Amplification (SDA) is another method of carrying out isothermal amplification of nucleic acids, which involves multiple rounds of strand displacement and synthesis, i.e., nick translation. A similar method, called Repair Chain Reaction (RCR), involves annealing several probes throughout a region targeted for amplification, followed by a repair reaction in which only two of the four bases are present. The other two bases can be added as biotinylated derivatives for easy detection. A similar approach is used in SDA. Target specific sequences can also be detected using a cyclic probe reaction (CPR). In CPR, a probe having 3′ and 5′ sequences of non-specific DNA and a middle sequence of specific RNA is hybridized to DNA that is present in a sample. Upon hybridization, the reaction is treated with RNase H, and the products of the probe identified as distinctive products that are released after digestion. The original template is annealed to another cycling probe and the reaction is repeated.

Still another amplification methods described in GB Application No. 2 202 328, and in PCT Application No. PCT/US89/01025, each of which is incorporated herein by reference in its entirety, may be used in accordance with the present disclosure. In the former application, “modified” primers are used in a PCR-like, template- and enzyme-dependent synthesis. The primers may be modified by labeling with a capture moiety (e.g., biotin) and/or a detector moiety (e.g., enzyme). In the latter application, an excess of labeled probes are added to a sample. In the presence of the target sequence, the probe binds and is cleaved catalytically. After cleavage, the target sequence is released intact to be bound by excess probe. Cleavage of the labeled probe signals the presence of the target sequence.

Other nucleic acid amplification procedures include transcription-based amplification systems (TAS), including nucleic acid sequence-based amplification (NASBA) and 3SR (Kwoh et al., 1989; Gingeras et al., PCT Application WO 88/10315, incorporated herein by reference in their entirety). In NASBA, the nucleic acids can be prepared for amplification by standard phenol/chloroform extraction, heat denaturation of a clinical sample, treatment with lysis buffer and minispin columns for isolation of DNA and RNA or guanidinium chloride extraction of RNA. These amplification techniques involve annealing a primer which has target specific sequences. Following polymerization, DNA/RNA hybrids are digested with RNase H while double stranded DNA molecules are heat denatured again. In either case the single stranded DNA is made fully double stranded by addition of second target specific primer, followed by polymerization. The double-stranded DNA molecules are then multiply transcribed by an RNA polymerase such as T7 or SP6. In an isothermal cyclic reaction, the RNAs are reverse transcribed into single stranded DNA, which is then converted to double stranded DNA, and then transcribed once again with an RNA polymerase such as T7 or SP6. The resulting products, whether truncated or complete, indicate target specific sequences.

Davey et al., EPO No. 329 822 (incorporated herein by reference in its entirety) disclose a nucleic acid amplification process involving cyclically synthesizing single-stranded RNA (“ssRNA”), ssDNA, and double-stranded DNA (dsDNA), which may be used in accordance with the present disclosure. The ssRNA is a template for a first primer oligonucleotide, which is elongated by reverse transcriptase (RNA-dependent DNA polymerase). The RNA is then removed from the resulting DNA:RNA duplex by the action of ribonuclease H (RNase H, an RNase specific for RNA in duplex with either DNA or RNA). The resultant ssDNA is a template for a second primer, which also includes the sequences of an RNA polymerase promoter (exemplified by T7 RNA polymerase) 5′ to its homology to the template. This primer is then extended by DNA polymerase (exemplified by the large “Klenow” fragment of E. coli DNA polymerase I), resulting in a double-stranded DNA (“dsDNA”) molecule, having a sequence identical to that of the original RNA between the primers and having additionally, at one end, a promoter sequence. This promoter sequence can be used by the appropriate RNA polymerase to make many RNA copies of the DNA. These copies can then re-enter the cycle leading to very swift amplification. With proper choice of enzymes, this amplification can be done isothermally without addition of enzymes at each cycle. Because of the cyclical nature of this process, the starting sequence can be chosen to be in the form of either DNA or RNA.

Miller et al., PCT Application WO 89/06700 (incorporated herein by reference in its entirety) disclose a nucleic acid sequence amplification scheme based on the hybridization of a promoter/primer sequence to a target single-stranded DNA (“ssDNA”) followed by transcription of many RNA copies of the sequence. This scheme is not cyclic, i.e., new templates are not produced from the resultant RNA transcripts. Other amplification methods include “RACE” and “one-sided PCR” (Frohman, 1990; Ohara et al., 1989; each herein incorporated by reference in their entirety).

Methods based on ligation of two (or more) oligonucleotides in the presence of nucleic acid having the sequence of the resulting “di-oligonucleotide,” thereby amplifying the di-oligonucleotide, may also be used in the amplification step of the present disclosure (Wu et al., 1989, incorporated herein by reference in its entirety).

### B. Intercalating Dyes

In biochemistry, intercalation is the insertion of molecules between the planar bases of deoxyribonucleic acid (DNA). This process is used as a method for analyzing DNA and it is also the basis of certain kinds of poisoning. There are several ways molecules (in this case, also known as ligands) can interact with DNA. Ligands may interact with DNA by covalently binding, electrostatically binding, or intercalating. Intercalation occurs when ligands of an appropriate size and chemical nature fit themselves in between base pairs of DNA. These ligands are mostly polycyclic, aromatic, and planar, and therefore often make good nucleic acid stains. Intensively studied DNA intercalators include berberine, ethidium bromide, proflavine, daunomycin, doxorubicin, and thalidomide. DNA intercalators are used in chemotherapeutic treatment to inhibit DNA replication in rapidly growing cancer cells. Examples include doxorubicin (adriamycin) and daunorubicin (both of which are used in treatment of Hodgkin's lymphoma), and dactinomycin (used in Wilm's tumur, Ewing's Sarcoma, rhabdomyosarcoma).

Metallointercalators are complexes of a metal cation with polycyclic aromatic ligands. The most commonly used metal ion is ruthenium(II), because its complexes are very slow to decompose in the biological environment. Other metallic cations that have been used include rhodium(III) and iridium(III). Typical ligands attached to the metal ion are dipyridine and terpyridine whose planar structure is ideal for intercalation.

In order for an intercalator to fit between base pairs, the DNA must dynamically open a space between its base pairs by unwinding. The degree of unwinding varies depending on the intercalator; for example, ethidium cation (the ionic form of ethidium bromide found in aqueous solution) unwinds DNA by about 26°, whereas proflavine unwinds it by about 17°. This unwinding causes the base pairs to separate, or “rise”, creating an opening of about 0.34 nm (3.4 Å). This unwinding induces local structural changes to the DNA strand, such as lengthening of the DNA strand or twisting of the base pairs. These structural modifications can lead to functional changes, often to the inhibition of transcription and replication and DNA repair processes, which makes intercalators potent mutagens. For this reason, DNA intercalators are often carcinogenic, such as the exo (but not the endo) 8,9 epoxide of aflatoxin B1 and acridines such as proflavine or quinacrine.

Intercalation as a mechanism of interaction between cationic, planar, polycyclic aromatic systems of the correct size (on the order of a base pair) was first proposed by Leonard Lerman in 1961. One proposed mechanism of intercalation is as follows: In aqueous isotonic solution, the cationic intercalator is attracted electrostatically to the surface of the polyanionic DNA. The ligand displaces a sodium and/or magnesium cation present in the “condensation cloud” of such cations that surrounds DNA (to partially balance the sum of the negative charges carried by each phosphate oxygen), thus forming a weak electrostatic association with the outer surface of DNA. From this position, the ligand diffuses along the surface of the DNA and may slide into the hydrophobic environment found between two base pairs that may transiently “open” to form an intercalation site, allowing the ethidium to move away from the hydrophilic (aqueous) environment surrounding the DNA and into the intercalation site. The base pairs transiently form such openings due to energy absorbed during collisions with solvent molecules.
Some of the most commonly used intercalating DNA dyes include:
Ethidium Bromide. This intercalating agent experiences a roughly 20-fold increase in brightness on binding to DNA and is excellent for staining DNA in agarose gel electrophoresis. It fluoresces under UV light.
SYBR® Green. This highly sensitive DNA stain fluoresces under ultraviolet light. Helixyte™ Green has the same spectral properties to those of SYBR® Green. Helixyte™ Green has much greater sensitivity for dsDNA, thus especially useful for assays where the presence of contaminating RNA or ssDNA might obscure results.
SYBR® Safe. The major advantages of this DNA dye are that it is as sensitive as ethidium bromide and can be visualized without UV light. A blue light box offers a safer option for visualization as the wavelengths do not cause DNA damage. SYBR® Safe was introduced as a safer alternative to EtBr and SYBR® Green, but unfortunately, it is much less sensitive than SYBR® Green. It only has sensitivity comparable to EtBr. Gelite™ Safe has been developed specifically to be less hazardous than EtBr for staining DNA in agarose and acrylamide gels with much higher sensitivity. Gelite™ Safe has greatly improved safety and uncompromised sensitivity.
Propidium Iodide. This intercalating DNA dye exhibits a 20 to 30-fold increase in fluorescence on binding to DNA. The dye is membrane impermeable and can only enter cells with compromised membranes, making it an excellent probe for identifying dead cells.
SYBR® Gold. Upon binding to nucleic acids, this highly sensitive intercalating DNA dye exhibits 1000-fold greater UV fluorescence.
Crystal Violet. This is a highly sensitive dye that is detectable in the visible range, which eliminates the risks of UV exposure. It intercalates with DNA in a similar manner as ethidium bromide but is less mutagenic. It is an excellent option for detection of nucleic acid in gel electrophoresis.
DAPI (4,6-diamidino-2-phenylindole). DAPI binds strongly to A-T rich regions of dsDNA. On binding it exhibits a roughly 20-fold increase in fluorescence. Its inability to pass through intact cell membranes easily makes DAPI more successful at staining dead cells or cells with compromised membranes rather than live cell staining.
7-AAD (7-aminoactinomycin D). 7-AAD binds strongly to the G-C rich regions of double-stranded DNA. Its large stokes shift makes it effective for use in multicolor analysis in conjunction with blue and green-fluorescent probes.
Gel Red. A robust, stable and sensitive DNA dye, Gel Red can be used as an in-gel stain or post stain. It is visualized using UV light.
IC Green/LC Green Plus. LC Green dyes are designed for high-resolution melting curve analysis to detect DNA sequence variants. The addition of these dyes tends to increase the melting temperature of DNA by 1-3° C., thus possibly requiring adjustment of cycling parameters. They are manufactured by Idaho Technology. They can detect heteroduplexes during melting analysis after PCR, are extremely stable, and do not inhibit PCR. LC Green PLUS is particularly useful in melting instruments with 96- or 384-well microtiter plates, has superb fluorescence intensity, and can be used with a variety of fluorescence-based PCR detection systems. LC Green I is a dsDNA binding dye used for Hi-Res Melting curve analysis and is particularly useful in HRM analysis to detect DNA sequence variants (SNPs, insertions/deletions).

### V. APPLICATIONS

The potential applications for the technology disclosed herein is vast and virtually unlimited except by the need or desire to distinguish nucleic acid sequence variation using relatively low resource tools.

One significant application of the technology is in the assessment of variation the causative agents of infectious disease. There is significant interest in assessing disease causing viral or bacterial variants that have different disease course/risk and varying susceptibility to treatment. SARS-CoV-2, HIV, malaria and tuberculosis infections are specifically considered, just to name a few.

Other potential applications include assessing specific genetic information about an individual without incurring the expense of complete genome sequencing. This might include the identification of critical sequence information required for pharmaceutical intervention, such as testing for known genetic variants associated with known adverse reactions to a drug. This could translate into companion diagnostic to guide treatments towards or away from certain subjects and to determine which diseases (cancers, infections) should be treated with which drugs.

For a discussion of anti-microbial resistance and human gene variations, see Tong et al., 2012.

A specific example of “companion” diagnostic application would be in conjunction with receptor tyrosine kinase genes in non-small cell lung cancer (NSCLC) patients (Paez et al., 2004) described somatic mutations of the epidermal growth factor receptor gene EGFR found in 15 of 58 unselected tumors from Japan and 1 of 61 from the United States. Treatment with the EGFR kinase inhibitor gefitinib (Iressa) causes tumor regression in some patients with NSCLC, more frequently in Japan. EGFR mutations were found in additional lung cancer samples from U.S. patients who responded to gefitinib therapy and in a lung adenocarcinoma cell line that was hypersensitive to growth inhibition by gefitinib, but not in gefitinib-insensitive tumors or cell lines. These results suggest that EGFR mutations may predict sensitivity to gefitinib.

A specific example of genetic screening for disease is sickle cell screening (Yue et al., 2014) compared complex sequencing methods to HRM methods for screening of sickle cell disease. Their study examined 511 individuals on the island of Bioko (Equatorial Guinea) and attempted to establish a method for rapid sickle cell disease screening. Following DNA extraction and polymerase chain reaction (PCR) amplification, high resolution melting (HRM) analysis was used to assess the specificity of fluorescence signals of the PCR products and to differentiate various genotypes of these products. The analytical results of HRM were validated using DNA sequencing. By HRM analysis, 80 out of 511 samples were classified as hemoglobin S (Hb S) heterozygotes, while 431 out of 511 samples were classified as wild-type. No mutant homozygote was identified. DNA sequencing indicated that within the 431 wild-type samples as indicated by HRM analysis, one case was actually a Hb S heterozygote and another case was a rare hemoglobin S-C genotype (sickle-hemoglobin C disease). One out of 80 suspected Hb S heterozygotes as indicated by HRM was confirmed as wild-type by DNA sequencing and the results of residual 508 cases were consistent for HRM analysis and sequencing. This suggests that HRM analysis is a simple, high-efficiency approach for Hb S screening and is useful for early diagnosis of SCD and particularly suitable for application in the African area.

In sum, the ease of use, simplicity, flexibility, low cost, nondestructive nature, superb sensitivity, and specificity of the present methods makes these logical tools of choice to screen DNA sequences in myriad of settings, including presequence screening, single nucleotide polymorphism (SNP) typing, methylation analysis, quantification (copy number variants and mosaicism), an alternative to gel-electrophoresis and clone characterization. See Vossen et al., 2009.

### VI. KITS

In still further embodiments, the present disclosure concerns kits for use with the methods described above. As the L-DNA will be used in all embodiments, the L-DNA will be included in the kit. The kits will thus comprise, in suitable container means, an L-DNA, and optionally reagents including dyes, labels, standards, detection devices, and instructions for using the same.

The L-DNA and other reagents may further comprise suitably aliquoted compositions. The kits may contain individual reagents or mixed reagents, in one or more separate containers. The components of the kits may be packaged either in aqueous media or in lyophilized form.

The containers of the kits will generally include at least one vial, test tube, flask, bottle, syringe or other container means, into which the antibody may be placed, or preferably, suitably aliquoted. The kits of the present disclosure will also typically include a means for containing the reagent containers in close confinement for commercial sale. Such containers may include injection or blow-molded plastic containers into which the desired vials are retained.

### VII. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

Preliminary data with EC77 base pair test region. Initial testing was performed with a double-stranded L-DNA that the inventor previously used to control PCR cycling (Tazawa et al., 1991). The sequence is almost the same length as the 81 bp RRDR region of TB, and the inventor already had a set of primers and a reaction design in place that works well. He used this existing 77 base pair double-stranded L-DNA sequence (Biomers, Germany) and obtained the D-DNA equivalent sequences. This E. coli 77 sequence is part of the uidA gene with the sequence: ACA AGA AAG GGA TCT TCA CTC GCG ACC GCA AAC CGA AGT CGG CGG CTT TTC TGC TGC AAA AAC GCT GGA CTG GCA TG (in many strains, e.g., E. coli strain MS1665; CP097721.1; SEQ ID NO: 2). The D-DNA target and primer sets for the E. coli 77 target were obtained from IDT. Reaction mixtures were formulated using Luna Universal qPCR Master Mix (New England BioLabs, Cat #E3003), which contains the intercalating dye SYBR® Green. Target was added at 2×10⁶ copies per reaction. A ThermoFisher Scientific QuantStudio 5 instrument was used to perform the PCR reaction and acquire the melt temperature data according to manufacturer's protocol. For melt analysis data collection was performed using the default settings on the QuantStudio 5. The QuantStudio 5 software determines derivative peak and then compares it to the instrument calibration standards to report a melt temperature. To minimize all but machine error, 96 identical reactions were run simultaneously. Using the QuantStudio 5 calibrated instrument, D-DNA and L-DNA wild-type sequences were shown as not different but single base mutations and 5 base mutations are significantly different (*) (ANOVA, unpaired t-test) from the wild-type sequence. The inventor will use this method to characterize the TB mutations described above and, under these ideal conditions, expect to find small but similar significant changes for reagents designed to mimic the variations expected in the TB RRDR region.

TABLE A
Calibrated instrument determination of melt temperature for E. coli
wild-type and mutant sequences (° C., mean ± s.d., N = 96).
Mutant Type

| Wild-Type | Wild-Type | 1 bp C→T | 1 bp T→C | 5 bp |
|---|---|---|---|---|
| (D-DNA) | (L-DNA) | mutant | mutant | mutations |
| 84.38 ± 0.13 | 84.50 ± 0.15 | 83.94 ± 0.13* | 85.00 ± 0.13* | 87.31 ± 0.15* |

Design the L-DNA comparator. A design to target the RRDR region, is based on the sequence from H37 (GenBank: L05910.1) given by GGC ACC AGC CAG CTG AGC CAA TTC ATG GAC CAG AAC AAC CCG CTG TCG GGG TTG ACC CAC AAG CGC CGA CTG TCG GCG CTG (SEQ ID NO: 1). This is the 81 bp sequence that is expected to be found and amplified in a patient sample infected with a strain susceptible to rifampicin treatment. L-DNA matching this sequence will be synthesized and added to PCR reactions to confirm that the susceptible sequence is present.

In a second example, the TB katG amplicon Mycobrowser sequence: H37Rv 1 Rv1908c 1 katG (Genbank sequence: KP746928.1) ATC TGG TCG GCC CCG AAC CCG AGG CTG CTC CGC TGG AGC AGA TGG GCT TGG GCT GGA AGA GCT CGT ATG GCA CCG GAA CCG GTA AGG ACG CGA TCA CCA GCG GCA TCG AGG TCG TAT GGA CGA ACA CCC CGA CGA AAT GGG ACA ACA GTT TCC TCG AGA TCC TGT ACG GCT ACG AGT GGG AGC TGA CGA AGA GCC CTG CT (SEQ ID NO: 3) will be synthesized in L-DNA. This is sequence that is expected to be found and amplified in a patient sample infected with a strain susceptible to isoniazid treatment. L-DNA matching this sequence will be synthesized and added to PCR reactions to confirm that the susceptible sequence is present and treatable with the TB drug isoniazid.

Optimize L-DNA melt template comparison method. The melt temperature differences will be detected by adding L-DNA to the PCR reaction, measuring the melt temperature before and after PCR, and then comparing the shifts in the derivatives of the pre-PCR and post-PCR melt analysis curves. The comparator L-DNA is added to a PCR reaction tube in low concentration along with the intercalating dye and an initial melt analysis curve is obtained. Importantly, before the PCR reaction has begun, the reaction does not contain any detectable double-stranded D-DNA amplicon product so this initial melt analysis only reflects the characteristics of the L-DNA wild-type comparator. After PCR has been completed, the post-PCR reaction now contains typically more than 10¹² copies of amplicon in D-DNA form. A second melt curve is then generated and compared to the initial melt curve obtained with the low concentration of the L-DNA wild-type comparator.

Select the appropriate intercalating dye. The principle of melt analysis is based on the observation that an intercalating dye increases in fluorescence when it inserts between two strands of a double-stranded DNA. At low temperatures, the intercalating dye produces a high fluorescence value when the two complementary strands are hybridized together. As the temperature is increased the strands separate and the fluorescence decreases. A sudden change in fluorescence is observed when the melt temperature is reached (Thermo Fisher Scientific Inc., 2019). For these applications, the inventor needed to identify an intercalating dye that fluoresces when intercalating within D-DNA and L-DNA and has a sufficiently high fluorescence signal. There are several commercially available intercalating dyes for measuring double-stranded DNA with fluorescence. The inventor has identified potential candidate intercalating dyes as LCGreen (Biofire Defense) (Zimmers et al., 2019), Invitrogen SYBR® Gold, Invitrogen SYTO 64 Red, Life Technologies SYTO 82 Orange, Invitrogen SYBR® Green I, Biotium EvaGreen, and Invitrogen/Molecular Probes SYTO 9 Green and others may become available (Schreiber-Gosche & Edwards, 2009). The inventor has observed that all currently available dyes intercalate into both D-DNA and L-DNA. For example, SYBR® green intercalates and can be used to detect the separation of two complementary strands as a function of temperature for D-DNA but also for L-DNA as well. L-DNA melting is observed with the intercalating dye SYBR® and appears to intercalate equally well in D-DNA and L-DNA importantly leading to melt temperature curves which have the same melt temperatures for both D-DNA and L-DNA. Although the analysis is incomplete, all available dyes to date appear to have this property. In preliminary experiments, the inventor started with EC77 L-DNA strand at 7.525×1011 copies per reaction, and evaluated the capabilities of SYBR® Gold, SYBR® Green, EvaGreen, SYTO 82, SYTO 64, SYTO 9, and LCGreen dyes. Of the dyes evaluated, Evagreen, SYTO 9, and LCGreen seemed most promising as they readily intercalated in L-DNA and produced consistent melt temperature peaks using standard methods. Based on these observations, the inventor plans to initially focus on LCGreen.

Determine the L-DNA concentration. The L-DNA concentration added to the tube as a comparator is important for two reasons. First sufficient copies need to be present to be detectable before the PCR reaction and, secondly, the copies need to be present in sufficiently low copy numbers as to not interfere with the analysis of the melt curve obtained after the PCR reaction. These are competing requirements and optimization will be performed to find the concentration that is detectable but does not interfere with post-PCR analysis. The underlying premise is that a positive PCR reaction will produce large numbers of D-DNA amplicons that far exceed the number of spiked L-DNA comparators added to the reaction. In a positive sample, large numbers of amplicons are always produced even when the cycle threshold is delayed. The number of copies is dependent on the limiting reagent present in the PCR reaction and typical primer concentrations are often set at 10¹³ copies/reaction. Another assumption, of course, is that the L-DNA comparators do not participate in the PCR reaction which the inventor verified in previous studies using L-DNA as optical sensors for cycling PCR (Tazawa et al., 1991; Hauser et al., 2006). In the initial intercalating dye studies, he used L-DNA strands at 7.5×10¹¹ copies per μL but have also verified that at concentration 10-fold less (7.5×10¹⁰) copies per μL, the then inventor was still able to determine the melt temperature of the EC77 L-DNA sequence using LC Green fluorescent dye and standard analysis methods (data not shown).

Develop melt signal analysis. With the goal of creating an analysis method that is not tied to a particular instrument, a generalized method to analyze data obtained will be employed. Data from the PCR instrument melt temperature measurements will be exported from the PCR instrument and using MATLAB R2022b fit with a spline interpolation, and the derivatives computed. Raw melt curve data from the pre-amplification L-DNA internal control and from the post-amplification D-DNA amplicon will be analyzed for the presence of mutations using a differential analysis approach. The inventor will perform spline interpolation to reduce signal noise and increase the resolution of the real-time melt data by 100 times. Derivatives from each set of interpolated data will be used to determine the greatest absolute change of the melt curves, which are indicative of the melting point. Mutations will be determined to be present by comparison between the L-DNA internal control melting point and the post-amplification D-DNA melting point rather than the instrumentation calibration point. Statistical differences among the types of polymorphisms in comparison to wild-type controls will be determined and used as a measure of the analytical sensitivity to sequence variation. In preliminary experiments, the inventor used this method on melt data obtained from his own melt curves using the wild-type and mutation reagents shown Table A. The instrumented exported fluorescence data as a function of the instrument's calibration is shown in the upper panel of FIG. 1. and the lower panel compares the shift observed after analysis. The x-axis units for the differential analysis in the lower panel are listed as arbitrary units because they do not depend on the temperature. Note that this method does not calculate a melt temperature for comparison to the values like those in Table A, but instead identifies difference between the unknown and the internal comparator. The differentiation method identified single base-pair mutations (C→T, Figure) and five base-pair (FIG. 1) in the 77-base E. coli sequence with a sensitivity of 100%. The method did not classify any wild-type sequences as mutants, suggesting a specificity of 100%. In larger replicate trials (n=17) wild-type E. coli targets and single-base C→T mutant E. coli targets, the difference produced between L-DNA internal control and mutant amplicon was statistically significant while the L-DNA and wild-type signals were not significantly different (paired t-test, alpha=0.05). These preliminary results suggest robust differentiation between mutant and wild-type sequences by a L-DNA-based melt analysis of fluorescence versus time data obtained during heating. This analysis did not depend on the actual temperatures of each data point but did depend on the instrument to increase temperature at a rate slow enough that data intervals could resolve the small changes between the mutant types. These data were obtained using the melt temperature control and analysis software available on the RotorGene® PCR instrument which has more flexible melt analysis controls and accessible melt analysis data. This instrument has performed well in past comparison testing (Wu et al., 2002; Spurlock et al., 2017). According to published reports, this instrument can perform at a temperature ramp rate of 0.017° C./s, somewhat slower than other available instruments (You et al., 2011).

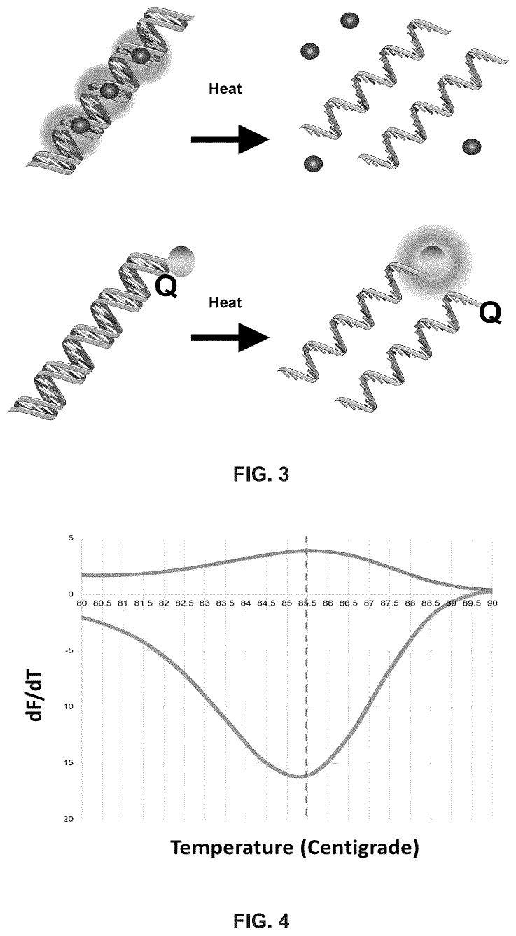

The inventor expects that the TB mutant-specific reagents will have small differences that can be detected with current well-calibrated instruments. He also expects that for these synthetic samples, the LCGreen intercalating dye, combined with fewer than 10¹⁰ copies of L-DNA as a wild-type comparator added to the pre-PCR reaction mixture, and melt curve derivative comparison without instrument calibration will detect mutations as well as or better than the calibrated instrument analysis methods. Alternate designs will be employed in the event that sensitivity to sequence variation is not high enough to detect single base mutations. Originally, the inventor planned to use an L-DNA comparator added to a PCR reaction and, along with a second intercalating dye, fluorescing in a different channel, simply measure the difference in the two fluorescence channels during melt analysis. However, upon further investigation two surprising, but consistent, findings were made: 1) available intercalating dyes appear to intercalate and fluoresce without regard to DNA chirality, and 2) measurement of melt temperature (Tₘ) using end-labelled L-DNA appears to be identical to the Tm obtained with some intercalating dyes (FIG. 4) suggesting another viable alternative to the methods described above. This is a slight variation on the L-DNA comparator approach. Instead of comparing pre-PCR and post-PCR using a single optical channel, the inventor will add a fluorophore to one end of the two L-DNA strands and add a quencher to the end of the complementary strand (FIG. 3). In this approach, the separation of the labelled L-DNA strands shows a decrease in the fluorescence signals associated with the melt of the double-stranded L-DNA. For example, the comparator L-DNA wild-type comparator used in the inventor's preliminary data was also labeled with a HEX fluorophore on one strand and a quencher on the opposite strand. With this comparator read in a second channel, this allows comparison between the HEX channel for L-DNA alone and the SYBR® green channel for all double-stranded structures. FIG. 4 illustrates the near concordance of the melt curves. The end-labelled L-DNA sequence had a calculated melt temperature of 85.5° C. for both channels by the RotorGene® software. If a chiral intercalating dye is identified, the inventor will explore that as a second alternative to the proposed method. In this second alternate method, the two intercalating dyes are read on separate optical channels and the derivatives in their melt curves are compared. Neither of these alternative methods requires a calibrated instrument. Single-base variation not detectable. Melt analysis methods are easier to implement for shorter sequences, mainly because SNP changes have a higher impact (Bentaleb et al., 2017). Initial PCR amplicons are expected to be in the 100 to 150 bp base pair range, but if this is too long, the inventor will shorten them to increase the observed variation in the melt curves.

### Example 2

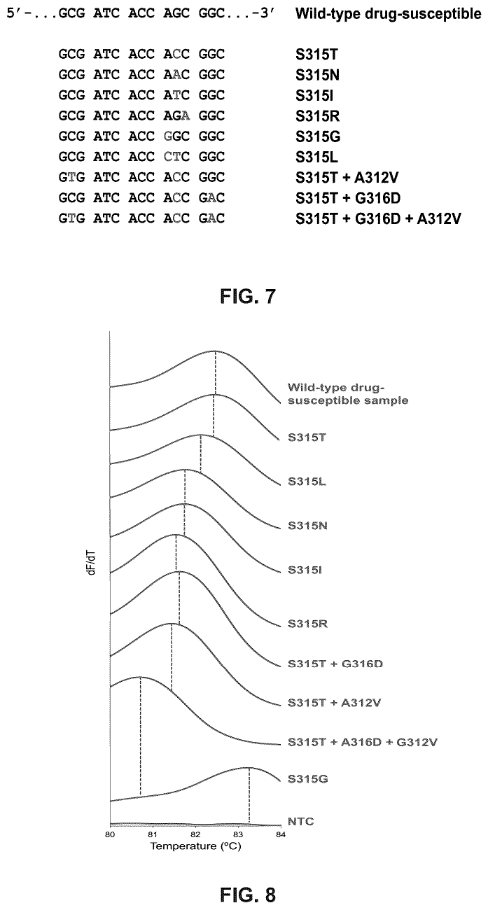

D-DNA Oligonucleotide Design. Single-stranded DNA targets representative of drug-susceptible katG (MycoBrowser, H37Rv: 2153889-2156111) and clinically relevant drug-resistant katG mutants were used as PCR targets. Mutants were selected to include a range of predicted melt differences from wild-type. A total of nine katG mutants with single and multi-base mutations conferring INH-resistance were tested, including the most prevalent variant S315T found in 94% of INH-resistant clinical isolates10,11 and other variants in the neighboring region (Vilcheze & Jacobs, 2014; WHO; Barozi et al., 2022; Yoon et al., 2012; Hazbón et al., 2006) (FIG. 7). The nine selected variants had a Tm spread of 2.43° C., offering easy and challenging classification cases against wild-type. All DNA oligonucleotides employed for development and testing of our assay were synthesized by Integrated DNA technologies (IDT, Skokie, IL).

Standard HRM Assay. The standard HRM approach for drug susceptibility screening uses a two-sample comparison of Tm's between an unknown PCR product and a known drug-susceptible PCR product. Reactions were performed in the Applied Biosystems™ QuantStudio™ 5 real-time PCR thermal cycler (Thermo Fisher Scientific #A28137). The QuantStudio™ 5 uses a 96-well format (Applied Biosystems™ #4483485) (Thermo Fisher Scientific Inc., 2019). Reactions included 10 μL of SensiFAST™ Probe No-ROX Kit (Bioline #BIO-86005), 2 μL of 10× LCGreen Plus (BioFire® Defense, LLC #BCHM-ASY-005), and 0.5 μL of 10 μM katG-specific primers (MEP176 and MEP177) for a final concentration of 250 nM each. Each target sample contained 2 μL of wild-type (MEP183) or mutant (MEP184-189,197-199) DNA target for a final concentration of 2×10⁶ copies per reaction. An example of a standard HRM reaction setup is outlined in Table B.

TABLE B

| Component | [Stock] | [Final] | Volume (μL) |
|---|---|---|---|
| Nuclease-free water | — | — | 5 |
| SensiFAST ™ Probe | 2× | 1× | 10 |
| No-ROX Kit | | | |
| LCGreen ® Plus | 10× | 1× | 2 |
| Forward primer | 10 μM | 250 nM | 0.5 |
| Reverse primer | 10 μM | 250 nM | 0.5 |
| Target | — | — | 2 |
| | | Total Volume | 20 |

In experiments testing the effect of heating variability, drug-susceptible and S315T reactions were loaded into mirrored quadrants of the 96-well plate. In experiments testing the effect of sample-to-sample salt concentration variability, salt-additive samples included 35 mM sodium chloride (Sigma Aldrich #S5150-1L) per reaction to simulate viral transport media equivalent salt contributions from possible extraction or culturing carryover. Salt-additive experiments assumed that thermal characteristics held constant plate-to-plate. Standard preparation and salt-additive drug-susceptible samples were loaded into two plates of matching well-to-well standard preparation versus salt-additive samples. PCR detection of the amplicon product was initiated with a 95° C. hold for 2 min followed by 40 cycles of 95° C. for 5 sec and 59° C. for 20 sec. Fluorescence was measured at the end of the annealing/extension step (59° C.). A high-resolution melt was performed immediately following PCR by annealing 95° C. to 50° C. at 0.1° C./sec followed by melting 65° C. to 95° C. at 0.025° C./sec. This melting ramp rate is often used in QuantStudio™ 5 HRM mutation scanning (Sun et al., 2023; Bentaleb et al., 2017; Koshikawa & Miyoshi, 2022; Miyoshi et al., 2022). Melt fluorescence was measured using a continuous acquisition mode. Double-stranded DNA PCR product fluorescence was monitored during PCR and the melt reaction using LCGreen Plus® on the green optical channel (excitation 470±15/emission 520±15). The QuantStudio™ 5 was initially factory-calibrated for optical and thermal accuracy (Thermo Fisher Scientific Inc., 2021). All standard calibration statuses (ROI/Uniformity, Background, Dyes) (Thermo Fisher Scientific Inc., 2021) were kept current. Custom dye calibration and custom melt curve calibration (Thermo Fisher Scientific Inc., 2021) were performed for LCGreen Plus (BioFire® Defense, LLC #BCHM-ASY-005).

Standard HRM Analysis and Statistics. PCR quantitation cycle (C_{q}) was determined with the QuantStudio™ 5 Design and Analysis Software. Non-amplifying samples did not report C_{q} and were excluded from the data analysis. Amplifying samples with C_{q} over 35 were excluded from the data analysis because they did not attain PCR amplification plateaus. Representative PCR amplification curves are included in Supplementary Materials. Derivative melting curve plots and Tₘ were calculated from QuantStudio™ 5 Design and Analysis Software. Significant differences of each variant as compared to wild-type were assessed within an experimental plate using unpaired t tests (of Tₘ) with a one-way ANOVA and Post-Hoc Bonferroni adjusted a threshold of 0.005 (n=3 trials with full PCR triplicates). Sample types were classified within each trial, that is drug-susceptible classification resulted when PCR product Tₘ's of a particular sample type were not significantly different from wild-type PCR product Tₘ's within a single 96-well plate. Since true positives are known, standard HRM was assessed for its sensitivity and specificity on 69 true drug-susceptible samples (n=6 trials) and 33 true S315T variant samples (n=4 trials) with a drug-susceptible Tₘ cut-off of 82.41° C. The true positive (TP, sensitivity) rate was calculated as the percentage of drug-susceptible (+) test results out of all true drug-susceptible (+) samples. The false negative (FN) rate was calculated as the percentage of S315T variant (−) test results out of all true drug-susceptible (+) samples. The false positive (FP) rate was calculated as the percentage of drug-susceptible (+) test results out of all true S315T (−) samples. The true negative (TN, specificity) rate was calculated as the percentage of S315T variant (−) test results out of all true S315T (−) samples. Heating variability testing was evaluated using significant differences (unpaired t test, significance level of α=0.95) of mirrored 96-well plate quadrants of S315T as compared to drug-susceptible wild-type Tₘ (n=1 trial with 24 replicates). Salt concentration variability testing was evaluated using significant differences (Wilcoxon signed-rank test, significance level of α=0.95) well-to-well of standard preparation as compared to salt-additive drug-susceptible wild-type Tₘ's (n=2 trials with 36 replicates). All statistics were performed in Microsoft® Excel 2022 except for the sensitivity and specificity analysis that was performed in MATLAB 2023A.

L-DNA-based HRM Assay. The L-DNA-based HRM for drug susceptibility screening uses a tₘ (elapsed melt time) comparison between an unknown PCR product and an internal drug-susceptible L-DNA comparator within a single sample. L-DNA-based HRM was tested using the same QS5 instrument, PCR cycling, PCR fluorescence monitoring, PCR quantification, melt reaction cycling, heating variability test setup, and salt concentration variability test setup as the standard HRM. Key changes from standard HRM are the inclusion of an additional reaction reagent (L-DNA), monitoring melt reaction fluorescence on a second optical channel, and different HRM analysis and statistics. Additional experimental designs for L-DNA-based HRM were performed to compare L-DNA and DNA PCR product melts, tune L-DNA strand concentration, and tune L-DNA strand ratio.

A double-stranded L-DNA drug-susceptible comparator was synthesized using left-helical enantiomeric DNA bases (i.e., L-DNA) (Anderson et al., 2007) such that it had the identical sequence and melt characteristics to the expected drug-susceptible katG DNA PCR product (FIG. 6)⁷. L-DNA does not interact with DNA when combined in a single reaction tube. L-DNA does not participate in or interrupt PCR (Tazawa et al., 1991; Hauser et al., 2006). The 56-base L-DNA was made to span a range of variants neighboring S315T and include the primer sequences within current synthesis limitations; IDT (Skokie, IL) cannot synthesize L-DNA longer than 100 nucleotide bases and Biomers.net (Germany) cannot synthesize L-DNA longer than 77 nucleotide bases. Increased L-DNA length enables coverage of more possible mutation sites but negatively affects single nucleotide polymorphism (SNP) discrimination power due to decreased melt temperature differences. That is, one SNP induces smaller melt temperature changes in longer sequences versus shorter sequences. The 56-base L-DNA was the same length as the DNA PCR amplicons. The 56 L-DNA was end-labeled with Texas Red (TXR) fluorophore and Black Hole Quencher 2 (BHQ2) quencher to monitor its behavior during melting on the orange fluorescent channel (excitation 580±10/emission 623±14). L-DNA fluorescent signal was scaled up by a factor of 18 in derivative melt plots for visual comparison with the PCR product's higher fluorescent signal. All L-DNA oligonucleotides employed for development and testing of our assay were synthesized by Biomers.net (Germany). L-DNA-based HRM reactions include 2 μL of L-DNA mix for final concentrations of 1×10¹¹ copies TXR-labeled forward strand L-DNA (23FEB_katGf56_TXR) and 3×10¹¹ copies BHQ2-labeled reverse strand L-DNA (23FEB_katG_56_Rcmp+5_BHQ2) per reaction. An example of L-DNA melt approach reaction setup is outlined in Table C.

TABLE C

| Component | [Stock] | [Final] | Volume (μL) |
|---|---|---|---|
| Nuclease-free water | — | — | 3 |
| SensiFAST ™ Probe | 2x | 1x | 10 |
| No-ROX Kit | | | |
| LCGreen ® Plus | 10x | 1x | 2 |
| Forward primer | 10 μM | 250 nM | 0.5 |
| Reverse primer | 10 μM | 250 nM | 0.5 |
| L-DNA mix | — | — | 2 |
| Target | — | — | 2 |
| | | Total Volume | 20 |

In experiments tuning L-DNA strand concentrations, reaction component deviations included 0.2 μL of 10 μM katG-specific primers for a final concentration of 100 nM each and 2 μL of L-DNA mix with final concentrations of 1×10¹¹, 2×10¹¹, and 4×10¹¹ copies of L-DNA strands (forward and reverse) per reaction. In experiments tuning L-DNA forward to reverse strand, reaction component deviations included 0.2 μL of 10 μM katG-specific primers for a final concentration of 100 nM each; 2 μL of L-DNA mix at 1:1, 1:2, and 1:3 ratios of forward to reverse strands at final L-DNA concentrations of 1×10¹¹ copies of forward strand plus 1×10¹¹, 2×10¹¹, and 3×10¹¹ copies of reverse strand.

L-DNA-based HRM Analysis and Statistics. Derivative melting curve plots and elapsed melt time (Tₘ) were calculated from the second degree Savitsky-Golay polynomials (Wittwer et al., 2003) at each point (performed in MATLAB 2023A). Elapsed melt time is a means of Tₘ reporting by via melting ramp time and fluorescence measurements during melting. Here, tₘ is defined as the elapsed melt time (in seconds) to reach the Tₘ. Time-defined melt analysis is utilized in L-DNA-based HRM to facilitate future studies in instruments with time-scale measurements and continuous heat ramping but do not incorporate temperature calibration. Significant differences of each PCR product as compared to L-DNA were assessed within a single sample using paired t tests (of tₘ difference) with a significance level of α=0.95 (n=3 trials with full PCR triplicates). Sample types were classified by technical replicate. Drug-susceptible classification resulted when sample tₘ difference fell within the 95% confidence interval (CI) for wild-type samples (n=3 trials with full PCR triplicates), defined experimentally as −2.68 sec to 0.35 sec. Since true positives are known, standard HRM was assessed for its sensitivity and specificity on 69 true drug-susceptible samples (n=6 trials) and 51 true S315T variant samples (n=5 trials) with a drug-susceptible tₘ difference cut-off of −3 sec. Heating variability testing was evaluated using significant differences (Mann-Whitney U test, significance level of α=0.95) of mirrored 96-well plate quadrants of S315T as compared to drug-susceptible wild-type (n=1 trial with 24 replicates). The Mann-Whitney U-test was performed for tₘ and tₘ difference. Salt concentration variability testing was evaluated using significant differences (Wilcoxon signed-rank test, significance level of α=0.95) well-to-well of standard preparation as compared to salt-additive drug-susceptible wild-type (n=2 trials with 36 replicates). The Wilcoxon signed-rank test was performed for tₘ and tₘ difference. Linear interpolation of three different L-DNA strand ratios was used to determine the relationship between copies of L-DNA reverse strands per reaction and L-DNA tₘ. The L-DNA reverse strand copy number with a tₘ matching that of wild-type PCR product was selected. All statistics were performed in Microsoft® Excel 2022.

High resolution melt analysis (HRM). HRM is a rapid technique for genotyping known variants and mutation scanning for unknown variants with, in some cases, discriminating between sequences with a single base difference (Dobrowolski et al., 2009; Wittwer, 2009; Erali et al., 2008). Polymerase chain reaction (PCR)-amplified nucleic acid products are most often characterized based on their sequence-specific hybridization melt temperature (Tₘ), as demonstrated by Wittwer et al. in 1997 (Wittwer et al., 1997). Mutation detection by HRM provides an alternative to the high-resource method of whole-genome sequencing. HRM is particularly advantageous in resource-limited settings due to its simple workflow, closed-tube mitigation of contamination risk, and instrument-enabled rapid target quantification. Many standard HRM assays classify samples based on a calibrated melt temperature comparison between PCR products. Methodological errors in the hybridization measurement produce significant changes in Tₘ that prevent classification of some mutations, particularly those with one base difference. For example, current top-of-the-line PCR instruments have heating variability across the 96-well heating block that limits Tₘ accuracy (Herrmann et al., 2007). Additional measurement error can be introduced into the system by sample-to-sample salt concentration variability resulting from reagent pipetting errors (e.g., individual reaction components or total reaction volumes) or kit-to-kit master mix differences (Liew et al., 2004).

In this example, the inventors hypothesize that including the same amount of double-stranded left-handed (L)-DNA (FIG. 6) (Sczepanski, 2021) in every sample provides a comparator hybridization event that reduces methodological errors affecting hybridization and more accurately detects melt changes in PCR products. They hypothesize that the L-DNA and PCR product hybridization events are affected by errors in the same way, and that the L-DNA to PCR product melt difference is sufficient to classify an unknown sample. In other words, given that the L-DNA hybridization event is constant, any change in the melt difference between L-DNA and the PCR product indicates that the PCR product has an unexpected sequence.

In settings where sequencing is unavailable, alternative screening methods for drug-susceptibility are desirable to match disease-infected patients with effective drug regimens. Accurate sequence validation is critical for successful drug-susceptibility testing. However, drug resistance-related mutations are often difficult to detect by HRM because their single or multi-nucleotide base changes induce very small melt shifts. Here, the inventors developed an HRM drug-susceptibility classification strategy that includes a comparator L-DNA in every sample. L-DNA-based HRM directly compares a drug-susceptible sequence L-DNA to an unknown D-DNA (DNA) PCR product within a single sample. The internal comparator L-DNA sequence was designed to match a drug-susceptible 56-nucleotide region of the consensus TB katG gene where over 250 INH-resistance-related mutations occur (Unissa et al., 2015). The L-DNA sequence spans the region containing the most prevalent variant S315T found in 94% of INH-resistant clinical isolates (Vilcheze et al., 2019; Zhang et al., 1992) and a subset of the many single or multi-base variants in the neighboring region that can also confer INH-resistance (Vilcheze & Jacobs, 2014; WHO; Barozi et al., 2022; Yoon et al., 2012; Hazbón et al., 2006). These variants have reported Tₘ differences from wild-type ranging from −2.0° C. (easier to detect) down to less than −0.2° C. (more difficult to detect). The melt difference between the L-DNA and the PCR product in each sample was used to successfully classify all samples as either drug-susceptible or not drug-susceptible. With further validation, the L-DNA-based approach offers a means to classify an unknown sample without any in-assay comparators.

In this example, the classification performance of the proposed L-DNA-based HRM method was compared to standard HRM for synthetic targets based on known sequence variants with melt differences ranging from very small (here −0.2° C.), characteristic of a single base change, to relatively large (here −2.0° C.), characteristic of a three-base change. All variant samples had lower derivative peak melt temperatures compared to the known wild-type samples except S315G which had a higher melt temperature (FIG. 8). Standard HRM correctly classified all drug-susceptible katG sequences and all but two variants, S315T and S315G. The approach failed to correctly classify the most difficult and most clinically prevalent variant S315T as not drug susceptible. Correct classification of three trials for standard HRM was (drug-susceptible) wild-type 3/3, (not drug-susceptible) S315T 0/3, S315N 3/3, S315I 3/3, S315R 3/3, S315G 2/3, S315L 3/3, S315T+G316D 3/3, S315T+A312V 3/3, and S315T+G316D+A312V 3/3. Unpaired t tests between S315T and wild-type within a single 96-well plate had p-values less than 0.005 across all three trials (0.163, 0.047, and 0.136), indicating that the standard melt approach falsely classified the most clinically prevalent katG variant S315T as a drug-susceptible wild-type. All other mutants were correctly classified as non-wild-type. Relationships between Tₘ differences are illustrated in FIG. 12, with Tₘ differences spanning −1.84±0.14° C. to 0.59±0.14° C. In particular, wild-type (green) and S315T (red) Tₘ have overlapping standard deviations which illustrates the failure of this standard method.

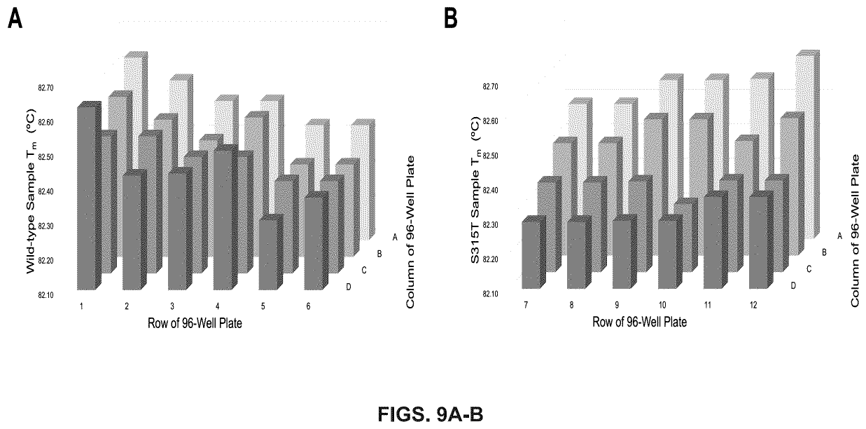

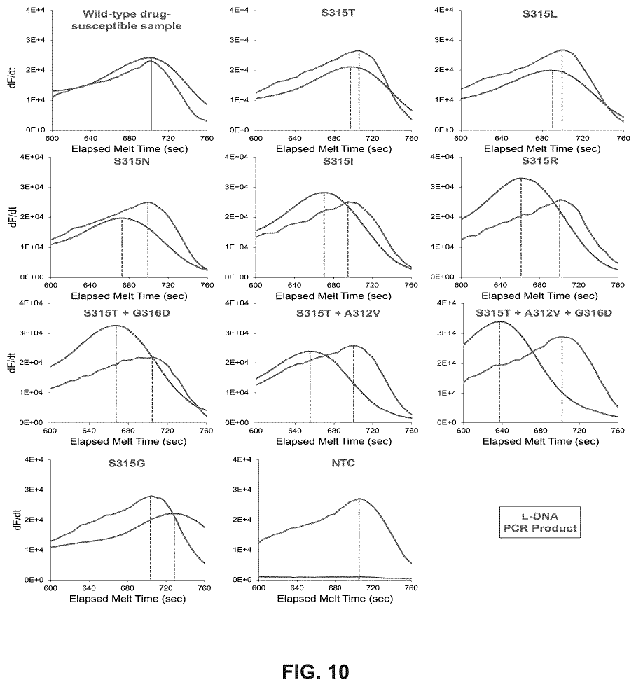

The inventors suspected that the S315T classification error was due to PCR instrument heating variability which has also been observed in other plate-based real-time PCR instruments (Herrmann et al., 2007). As expected, the direct test of the instrument's heating errors demonstrated heating variability across the 96 well plate which impacted standard HRM classification of the most difficult case, S315T. There was no significant difference between wild-type and S315T samples (p=0.132, p>0.05, unpaired t test, 24 replicates per sample type), indicating that standard HRM failed to distinguish between sample types and incorrect classification was more likely. 24 identical samples exhibited thermal edge effects of generally higher Tₘ's and zone-based Tₘ's for two samples in shared heating elements, with trends holding for both wild-type and S315T sample types (FIGS. 9A-B). The heating variability of the QuantStudio™ 5 (FIGS. 10A-B) is presumably due to variations in the instrument's six independent temperature heating zones (pairwise vertical columns) that cannot be corrected by the standard instrument calibration procedures.

Standard HRM's failed S315T test case was re-evaluated by L-DNA-based HRM to determine if sample-to-sample hybridization effects from non-uniform heating in this instrument could be overcome by adding L-DNA to every sample. Using tₘ differences between PCR product and L-DNA comparator in that sample, there was a significant difference between quadrants of drug-susceptible and S315T samples (p<0.05, Mann-Whitney U test, 24 replicates per sample type). This p-value of 2.626×10¹ indicated that the L-DNA-based HRM comparison correctly classified SNP S315T as not drug-susceptible. These data support our hypothesis, that within sample differences between the L-DNA and the PCR product can overcame the effect of heating variability that produce errors in sample Tₘ. Preliminary tests to determine if just adding L-DNA alone impacted this finding were done by using PCR product melt measurement alone for direct comparison to standard HRM reactions without L-DNA. Just adding L-DNA to samples did not change the results and standard melt analysis of these reactions did not correctly differentiate between wild-type and S315T. Specifically, there was no significant difference between wild-type and S315T when PCR product tₘ was used alone for sample differentiation (p=0.571, Mann-Whitney U test).

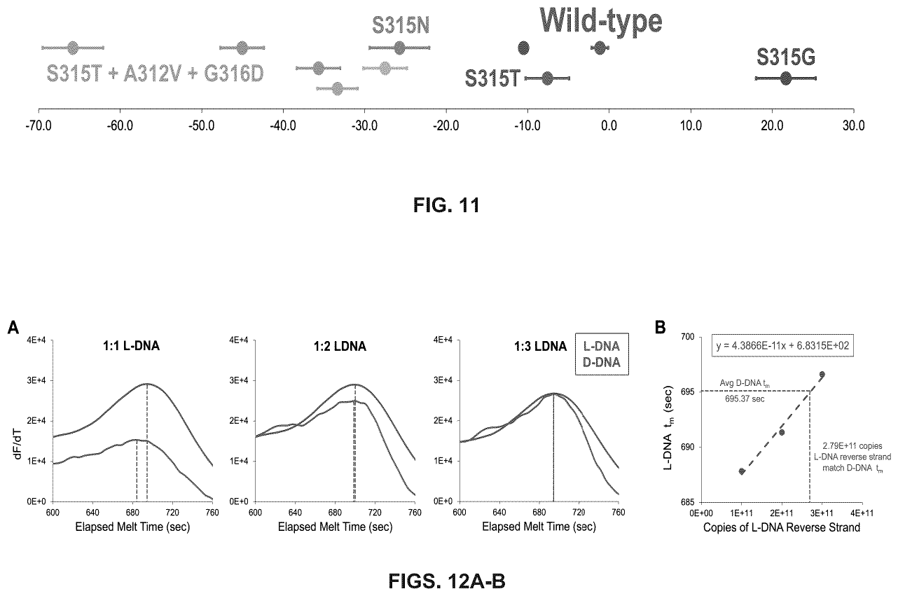

In tests of the L-DNA-based HRM method on the panel of all nine variants, L-DNA-based HRM correctly classified all wild-type and drug susceptibility variants. Correct classification of 9 replicates across three trials for L-DNA-based HRM was (drug-susceptible) wild-type 7/9, (not drug-susceptible) S315T 8/9, S315N 9/9, S315I 9/9, S315R 9/9, S315G 9/9, S315L 9/9, S315T+G316D 9/9, S315T+A312V 9/9, and S315T+G316D+A312V 9/9. L-DNA and DNA PCR product had nearly identical melt characteristics when the sequences matched (wild-type drug-susceptible katG) but differed if there was a sequence mismatch (katG variants) (FIG. 10). Across nine clinically relevant katG variant types, all drug-resistant samples except S315G had lower derivative peak elapsed melt times compared to the known drug-susceptible L-DNA internal comparator (FIG. 10). Average tₘ's and tₘ differences are reported in FIG. 11.

All nine katG variants had a significant difference between a given sample's PCR product and L-DNA tₘ's (p<0.05, paired t tests). Notably, the S315T p-value of 0.0003 demonstrated L-DNA-based HRM's enhanced detection resolution as compared to standard HRM's failed S315T discrimination. Variant tₘ differences spanned −66±4 sec to 22±4 sec across all sample types. In particular, wild-type (green) and S315T (red) have nonoverlapping average tₘ differences (±SD). Because the inventors had known sample classifications from other experiments (i.e., methodological error trials) performed in the same manner, L-DNA-based HRM S315T classification was further analysis using an expanded data set (69 true wild-type samples across n=6 trials; 51 true S315T samples across n=5 trials) was performed. L-DNA-based HRM performed at 80% sensitivity and 75% specificity when identifying wild-type samples against S315T with a drug-susceptible tₘ difference cut-off of −3 sec Table 1. For comparison's sake, the standard HRM data set was also expanded to include additional known sample classifications from other experiments (i.e., methodological error trials) performed in the same manner (69 true wild-type samples across n=6 trials; 33 true S315T samples across n=4 trials). Standard HRM performed at 83% sensitivity and 58% specificity when identifying wild-type samples against S315T with a drug-susceptible Tₘ cut-off of 82.41° C. Table 2. L-DNA-based HRM correctly classified this most difficult and most clinically prevalent variant S315T from wild-type sequences with 17% improved specificity over standard HRM. L-DNA-based HRM also decreased the false positive rate by 17% over standard HRM, indicating that L-DNA decreased the detrimental misdiagnosis of S315T variant samples as drug-susceptible resulting in continuation of ineffective treatment regimens and perpetuated disease transmission.

TABLE 1
Standard HRM*

| | | True Sample Classification | |
|---|---|---|---|
| | | Drug-susceptible | S315T Variant |
| | | (+) | (−) |
| Gold | Drug-susceptible | 83% TP | 42% FP |
| Standard | (+) | | |
| HRM Test | S315T Variant | 17% FN | 58% TN |
| Result | (−) | | |

*performed at 83% sensitivity and 58% specificity when classifying drug-susceptible wild-type katG samples among 69 true drug-susceptible samples (n = 6 trials) and 33 true S315T variant samples (n = 4 trials) with a drug-susceptible Tₘ cut-off of 82.41° C. (T = true, F = false, P = positive, N = negative)

TABLE 2
L-DNA-Based HRM

| | | True Sample Classification | |
|---|---|---|---|
| | | Drug-susceptible | S315T Variant |
| | | (+) | (−) |
| L-DNA | Drug-susceptible | 80% TP | 25% FP |
| HRM Test | (+) | | |
| Result | S315T Variant | 20% FN | 75% TN |
| | (−) | | |

*-performed at 80% sensitivity and 75% specificity when classifying drug-susceptible wild-type katG samples among 69 true drug-susceptible samples (n = 6 trials) and 51 true S315T variant samples (n = 5 trials) with a drug-susceptible tₘ difference cut-off value of −3 sec. (T = true, F = false, P = positive, N = negative)

The inventors identified heating variation as a major methodological error that reduced classification performance. While no other methodological errors were identified in our HRM testing, the inventors speculated that some types of sample preparation errors could introduce systematic hybridization changes which could also be corrected using L-DNA. For example, accidental viral transport media carryover from culturing or extraction, (Liew et al., 2004) kit-to-kit master mix differences or sample-to-sample salt concentration variability may be introduced by reagent pipetting errors (e.g., individual reaction components or total reaction volumes). Although not relevant to the standard HRM studies here, a contrived study of salt differences is included in supplementary materials to verify that a sample's salt content has a major potential impact on successful classification and that within assay differences overcome by L-DNA-based HRM restore classification capabilities.

In this proof-of-concept study, L-DNA-based HRM compared to standard HRM performed similarly for variants with melt characteristics that were easily detectable. The L-DNA based HRM performed better with the most difficult to classify case, S315T. In the context of susceptibility testing for INH resistance, this variant has melt characteristics very similar to the wild-type because it is produced by a single base change swap but it accounts for about 95% of the samples with are drug susceptible. L-DNA-based HRM reactions included the same amount of double-stranded L-DNA in every sample to provide a consistent comparator hybridization event that was used to reduce methodological errors affecting hybridization and more accurately detect melt changes in PCR products. As hypothesized, since L-DNA and the PCR product hybridization events were changed by heating and sample preparation errors in the same way, the L-DNA to PCR product melt difference corrected for any error-induced melt shifts. These experiments were designed to ensure that the L-DNA hybridization event was constant in every sample. Constant L-DNA concentration in every sample was maintained by adding L-DNA from a stock into the master mix. Analysis assumed that a change in the melt difference between L-DNA and the PCR product indicated that the PCR product had changed. Small mutation-induced melt shifts in the PCR product were detectable due to the reduction in errors and ultimately improved HRM classification over the standard method. Within these experiments a nearest neighbour synthetic was available, and statistical variance of the differences in the L-DNA and PCR product melt times was used to determine the classification cutoff. All samples with L-D differences less than this value are classified as drug susceptible and all samples with differences greater than this value are classified as not drug susceptible. Single 96-well plate experiments performed with a single master mix spiked with L-DNA, typically used three samples to determine this cutoff. Assuming correctly classified historical samples were collected using L-DNA-based HRM, this classification strategy may be used to classify a new unknown patient sample using just a single test sample. Achieving this assumes that the concentration of L-DNA is the same in every sample and that historical L-DNA-based HRM data exists for the samples used to set the cutoff (e.g., S315T).

A key design challenge for L-DNA-based HRM was determining how to discriminate between double-stranded L-DNA and D-DNA melting behavior in a single sample. All readily available intercalating dyes do not discriminate between enantiomeric DNA (data not shown). Therefore, if double-stranded L-DNA and double-stranded D-DNA are combined in a single reaction, the intercalation melt signal reports a composite melt curve. To overcome this L-DNA and D-DNA intercalating crosstalk, L-DNA was end-labeled with Texas Red. The goal was to measure only double-stranded DNA PCR product fluorescent signal on the green channel using LCGreen intercalating dye and measure only double-stranded L-DNA fluorescent signal on the orange channel using Texas Red fluorophore and quencher end-labeling. As FIG. 14 demonstrates, 4×1011 copies of double-stranded L-DNA per reaction produced detectable melt signals on both fluorophore-quencher (green) and intercalator (red) channels; however, 1×101 copies of double-stranded L-DNA per reaction produced sufficient fluorophore-quencher signal (green) for accurate L-DNA melt measurements with little crosstalk contribution detectable in the intercalator channel (red) (FIG. 14).

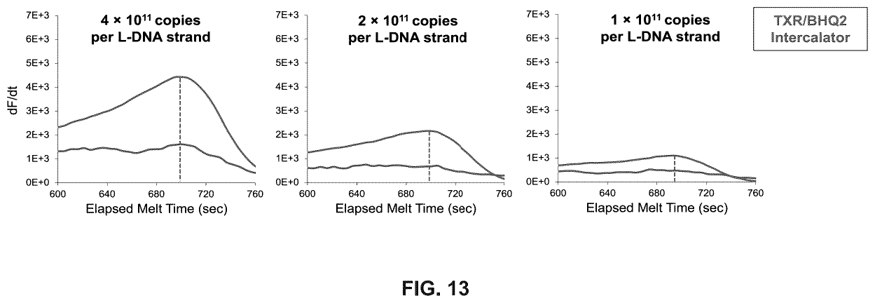

Additional design criteria were also important. The L-DNA used as the within sample comparison of L-DNA to unknown PCR product was synthesized to match the melt characteristics of the wild-type PCR product. Several factors made matching the melt characteristics of the two difficult. It is well known that end-labelling of the L-DNA sequence end-labeling used to overcome single tube detection also changes the DNA's melt temperature (Zimmers et al., 2019). Previous reports have also established that total DNA concentration and strand ratio also effect the melt temperature (Schreiber-Gosche & Edwards, 2009; Wu et al., 2002; Spurlock et al., 2017; You et al., 2011). In addition to these well-known factors, even when they have the same sequence, for unknown reasons, unlabeled double-stranded L-DNA and D-DNA have different melt temperatures measured by intercalation (data not shown). Since strand ratio was the easiest to adjust, the strand ratio of the L-DNA added was modified to compensate for these other factors. As FIGS. 13A-B demonstrate, different L-DNA forward-to-reverse strand ratios shift L-DNA tm. This phenomenon was used to compensate for all factors discussed above and achieve an empirical melt match between drug-susceptible L-DNA and D-DNA (right-most panel in FIG. 13A-B). As reverse strand L-DNA concentration increased, L-DNA tm measured via fluorophore-quencher signal increased and fluorophore-quencher signal increased in magnitude (FIG. 13A). The average drug-susceptible L-DNA tm per L-DNA ratio of 1:1, 1:2, and 1:3 was 688±11 sec, 691±8 sec, and 697±3 sec, respectively. A positive linear relationship between number of L-DNA reverse strand copies per reaction and L-DNA tm indicated that 2.79×1011 L-DNA reverse strand copies (and 1×1011 L-DNA forward strand copies) per reaction would produce the 695 sec tm matching average wild-type PCR product tm. (FIG. 13A-B). The optimal L-DNA strand ratio was rounded up from 1:2.79 to 1:3 (1×1011 L-DNA forward strand copies and 3×1011 L-DNA reverse strand copies per reaction) for ease of sample preparation in L-DNA-based HRM. This method produced a melt difference of approximately 1 sec between drug-susceptible L-DNA and wild-type PCR product (data not shown). In further support of melt matching, there was no statistical difference between tm's of drug-susceptible L-DNA and wild-type PCR product (p=0.169, paired t test, n=3 trials in full PCR triplicates). It is important to note that, although the inventors sought to match the L-DNA melt to the PCR product melt, it is not critical to do so. Even without tuning L-DNA, the melt difference will still be a constant in the system. L-DNA tuning is, in some sense, a reagent-based calibration that only needs to be completed one time in test development.

Notably, L-DNA-based HRM does not use temperature-based melt reporting determined by instrument calibration. Instead, L-DNA-based HRM utilizes time-defined melt analysis. Measurements were quantified using time instead of temperature for two reasons. First, any error in the temperature calibration of the QS5 instrument used in these studies will also contribute to errors in our analysis based on these values. And, secondly, this strategy facilitates future L-DNA-based HRM measurements in other types of real-time PCR instruments that are not as well-calibrated as the QS5 instrument. Time-defined melt analysis offers the potential for HRM sample classification in lower complexity real-time PCR instrumentation. At a minimum the real time PCR instrument is required to have melt analysis capabilities built into its software. This is very common as melt analysis is used for product assessment in many assays. Secondly, to perform analysis, all that is required is access to the sample times and fluorescence values recorded during the melt procedure.

If these features are available, the Nyquist Sampling Theorem and properties of the instrument can be used to estimate the limits to the L-DNA-based HRM capabilities of a particular instrument. The Nyquist sampling theorem states that in order to preserve the information in the original signal, the minimum sample frequency must be twice the maximum frequency of the signal. For the L-DNA based method the classification method depends on detecting melt differences between a wild-type sequence and a nearest melt neighbor within a single sample. For any given instrument with a known ramp rate and sampling frequency, this is given by 2 times the ramp rate (° C. Hz) times the sampling rate (fluorescence measurements in Hz). As an example, in experiments with the QS5 instrument which allows some freedom in how these values are set, the temperature ramp rate in Hz was set to 0.025° C./sec (“continuous mode”) and the fluorescence sampling rate was 1/3.3 sec or about 0.3 Hz (here determined by the “continuous mode”). This gives an estimated value of about 0.16° C. that can be resolved by L-DNA-base HRM. The value 0.16° C. is slightly better than the value of 0.2° C. that separates the wild-type from the nearest neighbor, S315T. This also helps to confirm that our choice of ramp rate and frequency rate comply with the requirements of the Nyquist theorem. In terms of instrument capabilities, the sampling rate maximum is likely the determining factor in machine implementation.

The known instrument melting ramp rate (i.e., 0.025° C./sec) can also be used to translate between temperature and time-based melt metrics. For example, the ˜1 sec time-based melt difference between drug-susceptible L-DNA and wild-type PCR product can be converted to the conventional temperature-metric melt difference of approximately 0.03° C. This conversion assumes any given sample has a constant conductive change in temperature with time.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
Adams et al., Anal. Chem. 89(1):728-735, 2016.
Anderson et al., Nucleoside, Nucleotides, Nucleic Acids, 3: 499, 1984.
Anderson et al., 2007.
Asseline et al., Nucleic Acids Res. 19: 4067-4074, 1991.
Barozi et al., ACS Omega. 7, 13313-13332 (2022).
Bentaleb et al., BMC Infect Dis 17, (2017).
Botezatu et al., Analytical Biochemistry. 419: 302-308, 2011.
Cui et al., Anal. Chem. 88: 1850-1855, 2016.
Davey et al., EPO No. 329 822
Dobrowolski et al., Hum Mutat. 30, 891-898 (2009).
Erali et al., Methods Mol Biol 429, 199-206 (2008).
Frohman, In: PCR Protocols: A Guide To Methods And Applications, Academic Press, N.Y., 1990.
Gingeras et al., PCT Application WO 88/10315
Hazbón et al., Antimicrob Agents Chemother 50, 2640 (2006).
Hauser et al. Nucleic Acids Res, 34(18):5101-11, 2006.
Herrmann et al., Clin Chem 53, 1541-1544 (2007).
Innis, et al., In: PCR Protocols. A Guide to Methods and Application, Academic Press, Inc. San Diego, 1990.
Koshikawa & Miyoshi, Biochem Biophys Rep; 31:101306, 2022.
Kwoh et al., Proc. Natl. Acad. Sci. USA, 86:1173, 1989.
Liew et al., Clin Chem, 50(7):1156-64, 2004.
Miller et al., PCT Application WO 89/06700
Miyoshi et al., Biochem Biophys Res Commun, 28; 606:128-134, 2022.
Narang et al., Methods Enzymol. 65: 610, 1980.
Ohara et al., Proc. Natl. Acad. Sci. USA, 86:5673-5677, 1989.
Paez et al., Science (304):1497-1500, 2004.
Reese, Tetrahedron 34: 3143, 1978.
Sambrook et al., In: Molecular cloning: a laboratory manual, 2^{nd} Ed., Cold Spring Harbor Laboratory Press, Cold Spring Harbor, N Y, 1989.
Schreiber-Gosche & Edwards, J Chem Educ 86, 644-650 (2009).
Sczepanski et al., The Glen Report 33, (2021).
Spurlock et al., Analytical Methods. 2017.
Sun et al., Microbiol Spectr 11, (2023).
Tazawa et al., Holy, A. Collect. Czech. Chem. Commun 113, 5843-5846 (1991).
Thermo Fisher Scientific Inc. QuantStudio 3 and QuantStudio 5 Real-Time PCR Systems. (2019).
Thermo Fisher Scientific Inc. QuantStudio 3 and 5 Real-Time PCR Systems Installation, Use, and Maintenance Guide. (2021).
Tong et al., J Clin Microbiol, 50(11): 3418-3421, 2012.
Unissa et al., Biomed Res Int. 2015:257983, 2015.
Urata et al., JACS 113: 8174-75, 1991.
Vilcheze, et al., Microbiol Spectr, 2(4):MGM2-0014-2013, 2014.
Vilcheze & Jacobs, Microbiol Spectr 2, (2014).
Vossen et al., Hum. Mutat. 30: 860-866, 2009.
Walker et al., Nucleic Acids Res., 20(7):1691-1696, 1992.
WHO. Global Tuberculosis Report 2023. (2023).
WHO. Catalogue of Mutations in Mycobacterium Tuberculosis Complex and Their Association
with Drug Resistance.
Wittwer et al., Biotechniques 22, 130-138 (1997).
Wittwer et al., Clin Chem 49, 853-860 (2003).
Wittwer, et al., Hum Mutat 30, 857-859 (2009).
Wu et al., Eur J Biochem 269, 2821-2830 (2002).
Yoon et al., Diagn Microbiol Infect Dis 72, 52-61 (2012).
You et al., Biopolymers 95, 472 (2011).
Young et al., Chen. Eur. J., 25: 7981-7990, 2019.
Yue et al., Molecular Medicine Reports 9: 2479-2484, 2014.
Zhang et al., Nature 358, 591-593 (1992).
Zimmers et al., Anal Methods, 14; 11(22):2862-2867, 2019.

1. A method of detecting sequence variation in a test D-DNA nucleic acid as compared to reference D-DNA sequence comprising:
(a) providing an L-DNA sequence that is identical to the reference D-DNA sequence except for its stereochemistry;
(b) obtaining a melt curve for said L-DNA sequence;
(c) obtaining a melt curve for said test D-DNA sequence under conditions identical to step (b); and
(d) comparing the melt curves of step (b) and step (c), wherein a difference in melt curves indicates that said test D-DNA does not have the same sequence as said reference D-DNA sequence.

2. The method of claim 1, wherein the L-DNA sequence and the test D-DNA sequence are detected using an intercalating dye and/or a dye/quencher pair or by hyperchromicity.

3. The method of claim 2, wherein the intercalating dye is selected from the group consisting of SYBR® Gold, SYBR® Green, EvaGreen, SYTO 82, SYTO 64, SYTO 9, and LCGreen dyes.

4. The method of claim 1, wherein obtaining a melt curve comprises heat separation of said L-DNA and/or said test D-DNA sequence.

5. The method of claim 1, wherein steps (b) and (c) are performed simultaneously in the same reaction mixture.

6. The method of claim 1, wherein said test D-DNA is a template dependent amplification product.

7. The method of claim 1, wherein multiple distinct L-DNA sequences with multiple melt properties are used together.

8. The method of claim 2, wherein the L-DNA is end-labeled one strand with a dye and end-labeled with quencher on the other strand, and the L-DNA and D-DNA is labeled with an intercalating dye that is not impacted by the quencher.

9. The method of claim 1, wherein the D-DNA is a template-dependent amplification product, such as wherein the L-DNA is present in template-dependent reaction mixture.

10. The method of claim 2, wherein a single intercalating dye is used for both L-DNA and D-DNA.

11. The method of claim 10, wherein the melt properties for the L-DNA are assessed prior to mixture with D-DNA, and the D-DNA is added to the L-DNA mixture and a second melt property assessment is performed.

12. The method of claim 1, wherein obtaining a melt curve for the L-DNA and/or the D-DNA comprises template dependent amplification.

13. The method of claim 1, further comprising quantitating D-DNA products.

14. The method of claim 1, wherein the L-DNA is one that has the same sequence as a natural D-DNA that is unique to a drug resistant pathogen, such as a drug resistant bacterium or virus.

15. The method of claim 1, wherein the melt temperature for L-DNA is adjusted to approximate the melt temperature of the D-DNA using the ratio of the forward:reverse strands of L-DNA.

16. The method of claim 15, wherein the pathogen is Mycobacterium tuberculosis, and the sequence is GGCACCAGCCAGCTGAGCCAATTCATGGACCAGAACAACCCGCT GTCGGGGTTGACCCACAAGCGCCGACTGTCGG CGCTG (SEQ ID NO: 1).

17. A kit comprising an L-DNA and at least one detectable moiety.

18. The kit of claim 17, wherein the L-DNA is a single-stranded sequence or a double-stranded sequence.

19. The kit of claim 17, wherein the L-DNA has the same sequence as a naturally occurring D-DNA sequence, or wherein the L-DNA has less than 10% base differences as compared to the naturally occurring D-DNA sequence.

20. The kit of claim 17, further comprising standard double-stranded sequences L-DNA and/or D-DNA sequence that melt at known relative temperatures.

21.-27. (canceled)
